(19) United States
(12) Patent Application Publication
(10) Pub. No.: US 2022/0273773 A1
(43) Pub. Date: Sep. 1, 2022

(54) TARGETING THE GASTROINTESTINAL BARRIER TO TREAT AGE-RELATED DISORDERS

(71) Applicant: The General Hospital Corporation, Boston, MA (US)

(72) Inventor: Richard A. Hodin, Newton, MA (US)

(21) Appl. No.: 17/631,218

(22) PCT Filed: Jul. 30, 2020
(86) PCT No.: PCT/US2020/044313

§ 371 Date: Jan. 28, 2022

Related U.S. Application Data
(60) Provisional application No. 62/994,577, filed on Mar. 25, 2020.
(60) Provisional application No. 62/881,967, filed on Aug. 2, 2019.

(51) Int. Cl. A61K 38/46; C12N 9/16; A61P 1/00

(52) U.S. Cl. CPC A61K 38/465; C12N 9/16; A61P 1/00; C12Y 301/03001

(57) ABSTRACT

The disclosure provides various aspects and embodiments of methods of, and compositions for use in methods of, treating age-related physiological alterations (e.g., those associated with age-related diseases or disorders) and/or treating or delaying the onset of age-related frailty in a subject. The methods include administering an AP-based agent or composition (e.g., a bovine intestinal alkaline phosphatase).

### TECHNICAL FIELD

The present disclosure relates to methods for treating age-related disorders and/or improving or delaying the onset of age-related frailty.

### CROSS-REFERENCE TO RELATED APPLICATIONS

This application claims the benefit of U.S. Provisional Application No. 62/994,577, filed on Mar. 25, 2020, and U.S. Provisional Application No. 62/881,967, filed on Aug. 2, 2019, the contents of both of which are hereby incorporated by reference in their entireties.

### DESCRIPTION OF TEXT FILE SUBMITTED ELECTRONICALLY

The contents of the text file submitted electronically herewith are incorporated herein by reference in their entirety: A computer readable format copy of the Sequence Listing (Filename: “MGH 25641_ST25.txt”; Date created: Jul. 23, 2020; File size: 43,814 bytes).

### BACKGROUND

Increased gastrointestinal permeability and chronic low-grade inflammation linked to persistent gastrointestinal-derived endotoxemia play a role in a variety of age-related diseases. Moreover, age-associated compositional changes of the gastrointestinal microbiota seem to interact with several physiological transitions and pathologies. Treatments targeting these age-related alterations are considered as potential approaches to slow the progression of or delay the onset of pathogenic deterioration and frailty associated with the aging and elderly populations. However, reliable interventions specifically targeting gastrointestinal barrier function and microbial composition are currently limited.

Alkaline phosphatase (“APs,” EC 3.1.3.1) is a hydrolase enzyme that can remove phosphate groups from various targets, including nucleotides and proteins. In particular, mammalian APs exert their properties by primarily targeting LPS (a TLR4 agonist), flagellin (a TLR5 agonist) and CpG DNA (a TLR9 agonist). APs also degrade intestine luminal Neurotoxic proteins (NTPs) (e.g., ATP, GTP, etc.), which promote the growth of good bacteria and reverses dysbiosis.

Given the paucity of agents directed to targeting gastrointestinal barrier function and microbial composition, thus extending lifespan and reducing or delaying age-related frailty, there is a need for reliable advancements that preserve and treat alterations of, or related to, intestinal homeostasis.

### SUMMARY

Accordingly, the present disclosure provides, in certain aspects, methods of and compositions for treating, e.g., improving, diminishing, attenuating, reducing, slowing the progression of, and/or delaying the onset of age-related physiological alterations of, or related to, intestinal homeostasis by administering an alkaline phosphatase (AP)-based agent to a subject in need thereof. In certain embodiments, the methods include determining whether the subject has an age-related physiological alteration.

In some embodiments, the age-related physiological alteration is selected from one or more of increased gastrointestinal permeability, increased gastrointestinal-derived systemic inflammation, increased chronic inflammation, increased gastrointestinal barrier dysfunction, dysbiosis, endotoxemia, and increased levels of proinflammatory cytokines or chemokines in the gastrointestinal tract and/or systemic circulation. The present disclosure contemplates that the age-related physiological alterations can be associated with frailty and/or a decreased lifespan. In further embodiments, the age-related physiological alterations are associated with an age-related disease or disorder and/or the subject is afflicted with the age-related diseases or disorders. For example, in certain embodiments, the age-related disease or disorder is selected from kidney failure, liver inflammation, steatosis, hepatic steatosis, non-alcoholic fatty liver disease (NAFLD), non-alcoholic steatohepatitis (NASH), type 2 diabetes, hepatocellular carcinoma, atherosclerotic cardiovascular disease (ASCVD), cachexia, metabolic syndrome, osteoarthritis, inflammatory bowel disease (IBD), and Alzheimer's disease.

In another aspect, the disclosure provides methods of and compositions for use in improving, treating, diminishing, attenuating, reducing, slowing the progression of, and/or delaying the onset of frailty, e.g., age-related frailty, by administering an AP-based agent to a subject in need thereof. In certain embodiments, the methods include determining whether the subject has a frailty, e.g., an age-related frailty.

In some embodiments, frailty is age-related. In some embodiments, frailty comprises an accumulation of deficiencies in major physiological functions, reduction of regeneration capabilities, impaired wound healing, and/or increased risk of age-related diseases. For example, in some embodiments, frailty is associated with natural aging or accelerated aging. Frailty can be measured according to any number of indices or tests known to one of skill in the art. For example, one such index, the Physiological Frailty Index (PFI), includes measurement of one or more parameters selected from grip strength, systolic blood pressure, diastolic blood pressure, blood flow volume, number of blood neutrophils, percentage of blood neutrophils, number of blood monocytes, percentage of blood monocytes, number of lymphocytes, number of red blood cells, hemoglobin levels, hematocrit levels, mean corpuscular volume, mean corpuscular hemoglobin levels, mean corpuscular hemoglobin concentration, and keratinocyte-derived cytokine levels. Deviation from a reference standard in any one individual is known as a deficit, and the overall average PFI score of the individual is a ratio of deficits to the total number of parameters measured.

In some embodiments, the present disclosure provides methods of and compositions for improving, treating, diminishing, attenuating, reducing, slowing the progression of and/or delaying the onset of frailty in a patient, as measured by a reduction in the PFI score of the patient. In some embodiments, methods and compositions of the present disclosure for improving, treating, diminishing, attenuating, reducing, and/or delaying the onset of frailty in a subject, e.g., a human or animal patient, include maintaining a PFI score over time so that the score increases at a rate slower than if the subject were not being administered the AP-based agents disclosed herein. In some embodiments of the present disclosure, the PFI score of the subject remains nearly the same over time. In further embodiments, methods of the present disclosure provide for an increase in cellular autophagy associated with natural aging and/or accelerated aging.

In another aspect, the disclosure provides methods of and compositions for use in improving, treating, diminishing, attenuating, reducing, slowing the progression of, and/or delaying the onset of age-related changes of gastrointestinal microbiota phylum diversity by administering an AP-based agent to a subject in need thereof. Specific microbiota phyla include, but are not limited to, Proteobacteria, Actinobacteria, Epsilonbactareota, Deferribacteres, Tenericutes, and Verrucomicrobia. In certain embodiments, the methods include determining whether the subject has any age-related changes of gastrointestinal microbiota phylum diversity.

In specific embodiments, methods and compositions of the present disclosure include improving, treating, diminishing, attenuating, reducing, slowing the progression of, and/or delaying the onset of accelerated aging. In some embodiments, accelerated aging is a progeroid syndrome or symptom thereof, including, but not limited to, Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), or restrictive dermopathy (RD). Subjects having one of these diseases or disorders typically have reduced longevity (i.e., a reduced lifespan).

In various embodiments, the AP-based agent is administered as a supplement, for example as a food additive. The AP-based agent can be administered chronically to the subject, e.g., for at least one year, or at least two years, or at least three years, or at least four years, or at least five years, or for the entirety of the subject's lifespan. The AP-based agent (e.g., IAP) can be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

The foregoing features of embodiments will be more readily understood by reference to the following detailed description, taken with reference to the accompanying drawings, in which:

FIGS. 1A-D depict sequences pertaining to alkaline phosphatase-based agents used in methods and compositions described herein.

FIGS. 2A-I depict how the age-dependent decline of IAP activity is paralleled by gastrointestinal barrier dysfunction and systemic inflammation. FIG. 2A shows IAP activity in human ileal contents (n=60) and FIG. 2B shows IAP activity in stool and ileal content of young and old wildtype (WT) mice measured by p-Nitrophenyl Phosphate (pNPP) assay. FIG. 2C depicts gastrointestinal permeability of IAP-knock out (KO) and WT mice as measured by serum FITC-dextran. Ileal tight junction protein mRNA expression levels for Occludin (FIG. 2D) and ZO-1 (FIG. 2E) were measured in mice and normalized by Bactin and measured by qPCR. Ileal Tnfa (FIG. 2F) and IL-6 (FIG. 2G) mRNA levels were also measured by qPCR. FIG. 2H depicts systemic serum tumor necrosis factor (TNF) levels measured by ELISA, and FIG. 2I shows systemic serum endotoxin levels measured by a limulus amebocyte lysate (LAL) assay. Each group included 5 animals, and data are representative of 3 biological replicates. Comparisons were made using Pearson's correlation analysis, unpaired Student t tests, or ANOVA with multiple-comparisons post hoc tests (Tukey's HSD). *P<0.05, **P<0.01, ***P<0.001, ****P<0.0001. In each set of two histograms of FIGS. 2C-I, the left bar is WT and the right bar is IAP-KO.

FIGS. 3A-F show a lack of IAP is associated with severe aging-related liver inflammation and an increased proinflammatory characteristic of portal serum. FIG. 3A-B depicts liver Tnfa (FIG. 3A) and IL-6 (FIG. 3B) mRNA levels measured by qPCR. FIG. 3C depicts the liver macrosteatosis score (Macrosteatosis 0%-5%, grade 0; 5%-33%, grade 1; 33%-66%, grade 2; 66%-100%, grade 3), while FIG. 3D shows Oil Red O staining of the liver (magnification, 20×). FIG. 3E shows portal serum endotoxin levels measured by a LAL assay, and FIG. 3F depicts Tnfa mRNA levels of primary mouse bone marrow (BM)-derived macrophages incubated with defined systemic or portal serum for 24 hours. Each group included 5 animals, and data are representative of 3 biological replicates. *P<0.05, **P<0.01, ***P<0.001, ****P<0.0001, as measured by ANOVA with multiple-comparisons post hoc tests (Tukey's HSD). In each set of two histograms of FIGS. 3A-C, E-F, the left bar is WT and the right bar is IAP-KO.

FIGS. 4A-C show how IAP supplementation extends lifespan in mice. FIG. 4A depicts survival of IAP-KO, WT, and IAP-supplemented mice (20 WT, 14 IAP-KO, and 6 IAP-supplemented mice). In FIG. 4A, the line showing 0% survival at 22 months represents the IAP-KO cohort; the line showing 0% survival at 30 months represents the WT cohort; and the line showing 0% survival at 36 months represents WT+IAP cohort. FIG. 4B-C shows the clinical frailty index of WT and IAP-KO mice (FIG. 4B) and vehicle- or IAP-supplemented WT mice (FIG. 4C). Unpaired Student t tests or ANOVA with multiple comparisons post hoc tests (Tukey's HSD) were used as statistical tests. Survival data were compared using the log-rank significance test. *P<0.05, **P<0.01. In each set of two histograms of FIGS. 4B-C, the left bar is WT and the right bar is IAP-KO.

FIGS. 5A-G show the effects of long-term IAP supplementation on aging-induced gastrointestinal barrier dysfunction and chronic systemic inflammation. FIG. 5A depicts gastrointestinal permeability as measured by systemic serum FITC-dextran 4 hours after oral gavage in 21-month old WT mice supplemented with vehicle or IAP for 11 months. FIG. 5B depicts blood serum endotoxin levels in WT vehicle- or IAP-supplemented mice as measured by LAL assay. FIG. 5C-E shows systemic serum IL-6, IL-1B and TNF-α levels in vehicle- or IAP-supplemented mice as measured by ELISA. FIG. 5F depicts fecal Lipocalin 2 (Lcn2) levels as measured by ELISA, and FIG. 5G shows fecal endotoxin levels as measured by LAL assay. Each group included 6 animals. Data expressed as mean ±SEM. Unpaired Student t tests are used as statistical tests. *P<0.05, **P<0.01, ***P<0.001. In each set of two histograms of FIGS. 5A-G the left bar is vehicle and the right bar is IAP.

FIGS. 6A-H depict how long-term IAP supplementation leading to an improved metabolic profile in aging mice. FIG. 6A shows serum total cholesterol levels in 18-month-old WT mice supplemented with vehicle or IAP for 8 months. FIG. 6B depicts serum triglyceride levels; FIG. 6C serum LDL-C levels; FIG. 6D serum HDL-C level; FIG. 6E blood urea nitrogen levels; FIG. 6F blood glucose levels; and FIG. 6G-H serum liver enzyme levels of AST and ALT. Each group included 6 animals. Data expressed as mean ±SEM. Two-tailed unpaired Student's t tests were used. *P<0.05, **P<0.01, ***P<0.001, ****P<0.0001. In each set of two histograms of FIGS. 6A-H, the left bar is vehicle and the right bar is IAP.

FIGS. 7A-H show how long-term IAP supplementation inhibits age-induced microbiome dysbiosis. FIG. 7A-B shows the Principal Coordinates Analysis (PCoA) of stool microbiome at the phyla level in IAP- and vehicle-treated mice at each time point (before treatment and 6 months after treatment). Relative abundances of different bacterial phylum are also depicted: Proteobacteria (FIG. 7C), Actinobacteria (FIG. 7D), Epsilonbacteraeota (FIG. 7E), Deferribacteres (FIG. 7F), Tenericutes (FIG. 7G), and Verrucomicrobia (FIG. 7H) in the stool of WT mice before and after supplementation with vehicle or IAP for 6 months. Each group included 6 animals. The data are averages ±SEM. *P<0.05, **P<0.01, ***P<0.001, by a PERMANOVA and 2-way ANOVA with a Bonferroni's multiple comparisons test. In each set of two histograms of FIGS. 7C-H, the left bar is vehicle and the right bar is IAP

Like reference symbols in the various drawings indicate like elements.

### DETAILED DESCRIPTION

The inventors discovered, inter alia, that, with age, the gut barrier becomes more dysfunctional, there is a loss of diversity in the gut microbiome, and there is an increase in inflammatory mediators in the systemic circulation. These physiologic changes accelerate the progression of age-related diseases and increase frailty. Additionally, the inventors discovered, inter alia, that endogenous levels of alkaline phosphatase decrease with aging. The inventors also discovered, inter alia, the absence of intestinal alkaline phosphatase exacerbates these physiologic changes. Importantly and unexpectedly, the inventors have discovered, inter alia, that even with normal levels intestinal alkaline phosphatase, supplementation with additional intestinal alkaline phosphatase dramatically attenuates these physiologic changes of aging. Administration of intestinal alkaline phosphatase maintains the gut microbiome diversity, maintains the gut barrier function, diminishes chronic low-grade systemic inflammation, improves the metabolic profile, protects the kidney, and dramatically extends lifespan.

Some of the aspects and embodiments of this disclosure are based, at least in part, on the finding that AP-based agents can be effective, for example, in improving, treating, diminishing, attenuating, reducing, slowing the progression of, and/or delaying the onset of age-related physiological alterations of, or related to, intestinal homeostasis and/or improving, treating, diminishing, attenuating, reducing, slowing the progression of, and/or delaying the onset of age-related frailty and/or improving, treating, diminishing, attenuating, reducing, and/or delaying the onset of age-related diseases or disorders.

Without wishing to be bound by theory, the methods and compositions disclosed herein are based, at least in part, on the discovery that an age-dependent decline in IAP levels contributes to the aging process given increased gastrointestinal permeability and reduced expression levels of tight junction proteins. Specifically, the present methods and compositions are based, in part, on the discovery that supplementation with IAP can improve, reduce, treat, diminish, attenuate, slowing the progression of, and/or delaying the onset of and even reverse the physiological alterations of intestinal homeostasis that are associated with aging and age-related diseases or disorders.

The aging process is manifested by a gradual accumulation of deficiencies in all major physiological functions, reduction of regeneration capabilities, and impaired wound healing, and increased risk of age-related diseases or disorders such as cancer, diabetes type 2, osteoarthritis, Alzheimer and Parkinson diseases, atherosclerosis and others. Cumulatively, all these events can be described as a gradual increase in frailty and measured by a so-called “frailty index.”

Aging is a gradual systemic pathological transformation of mammalian organism advancing with time, and is associated with accumulation of multiple deficiencies in functions of multiple organs and tissues and reduced regeneration capabilities leading to development of age-related chronic diseases or disorders including atherosclerosis, diabetes, pulmonary fibrosis, blindness, dementia, kidney dysfunction, osteoarthritis, and low grade chronic sterile inflammation as well as other age-related diseases and disorders contemplated herein. These conditions frequently coincide with a gradual development of geriatric syndromes including frailty, cognitive impairment and immobility. Aging is a natural and unavoidable process. Underlying causes of aging are still disputable; however, two features of aging are generally accepted as universal: an increase in DNA damage and development of systemic sterile chronic inflammation, both considered as major contributors of age-related pathologies.

### Physiological Alterations of Intestinal Homeostasis and Age-Related Diseases and Disorders

The present disclosure provides methods involving administering an AP-based agent to a subject to improve, treat, diminish, attenuate, reduce, slow the progression of, and/or delaying the onset of an age-related physiological alteration of, or that is related to, intestinal homeostasis.

For example, in some embodiments, the methods provided herein improve, treat, diminish, attenuate, reduce, slow the progression of, and/or delay the onset of age-related physiological alterations of intestinal homeostasis selected from one or more of increased gastrointestinal permeability, increased gastrointestinal-derived systemic inflammation, increased chronic inflammation, increased gastrointestinal barrier dysfunction, dysbiosis, endotoxemia, and increased levels of proinflammatory cytokines or chemokines. Examples of proinflammatory cytokines include, but are not limited to Interleukin 6 (IL-6), Tumor Necrosis Factor-alpha (TNF-α), Interleukin 1 (IL-1), Interleukin 8 (IL-8), and Interleukin 18 (IL-18). Examples of proinflammatory chemokines include, but are not limited to, C-Reactive Protein (CRP) and Macrophage-Derived Chemokine 2 (MDC-2).

In various embodiments, the age-related physiological alteration of intestinal homeostasis is measured by a decrease in ZO-1 protein, ZO-2 protein, occludin, or tight junction proteins, or is measured by an increase in HMGB1 (High Mobility Group Box 1).

In some embodiments, the age-related physiological alteration of intestinal homeostasis is associated with an age-related disease or disorder and the subject is afflicted with said age-related disease or disorder. Illustrative examples of age-related diseases or disorders that are contemplated by the present disclosure include, but are not limited to, kidney failure, liver inflammation, steatosis, hepatic steatosis, non-alcoholic fatty liver disease (NAFLD), non-alcoholic steatohepatitis (NASH), type 2 diabetes, hepatocellular carcinoma, atherosclerotic cardiovascular disease (ASCVD), cachexia, metabolic syndrome, osteoarthritis, inflammatory bowel disease (IBD), and Alzheimer's disease.

In embodiments, the age related disease is a renal disease or disorder. Renal function decreases with age. In embodiments, the AP agent reduces and/or slows the age-related increase in the level in the serum of blood urea nitrogen (BUN) and/or creatinine. In embodiments, the AP reagent slows the age-related decrease in creatinine clearance. In embodiments, the AP agent modulates a BUN-to-creatinine ratio. In embodiments, the age related disease is one of pre-renal azotemia, pre-renal failure, primary renal azotemia, acute or chronic kidney failure, and post-renal azotemia. In embodiments, the AP agent reduces or prevents loss in renal function associated with aging.

In various embodiments, the present disclosure provides methods and compositions for improving, treating, diminishing, attenuating, reducing, slowing the progression of, and/or delaying the onset of age-related physiological alterations of intestinal homeostasis in a non-elderly subject, wherein the method includes: screening the subject for one or more age-related physiological alterations of intestinal homeostasis selected from one or more of increased gastrointestinal permeability, increased gastrointestinal-derived systemic inflammation, increased chronic inflammation, increased gastrointestinal barrier dysfunction, dysbiosis, endotoxemia, and increased proinflammatory cytokines or chemokines, and wherein the subject is administered an AP-based agent if the screen indicates the physiological alterations are associated with aging. In specific embodiments, the screen for the one or more age-related physiological alterations is selected from a decrease in ZO-1 protein, a decrease in ZO-2 protein, a decrease in occludin, a decrease in tight junction proteins, and an increase in HMGB1 (High Mobility Group Box 1).

In some embodiments, the age-related physiological alteration of intestinal homeostasis is associated with frailty and/or a decreased lifespan.

For example, in some embodiments, the methods and compositions provided herein are used for improving, treating, diminishing, attenuating, reducing, slowing the progression of, and/or delaying the onset of age-related diseases or disorders such as Alzheimer's disease, type II diabetes, macular degeneration, chronic inflammation-based pathologies (e.g., arthritis), and/or to improve, treat, diminish, attenuate, reduce, slow the progression of, and/or delay the onset of development of cancer types known to be associated with aging (e.g., prostate cancer, melanoma, lung cancer, colon cancer, etc.), and/or with the purpose to restore function and morphology of aging tissues (e.g., skin or prostate), and/or with the purpose to improve morphology of tissue impaired by accumulated senescent cells (e.g., cosmetic treatment of pigmented skin lesions), and/or with the purpose to improve the outcome of cancer treatment by radiation or chemotherapy, and/or with the purpose to prevent recurrent and metastatic disease in cancer patients by elimination of dormant cancer cells. The disclosure is suitable for prophylaxis and/or therapy of human and non-human animal diseases and aging and age-related disorders.

In various examples, the disclosure relates to methods of and compositions for use in treating an individual suspected of having or at risk for developing an age-related disease or disorder, including but not necessarily limited to Alzheimer's disease, Type II diabetes, macular degeneration, or a disease comprising chronic inflammation, including but not necessarily limited to osteoarthritis.

In some embodiments, the methods and compositions described herein provide for treatment of a patient identified as having or at risk of having one or more of a cardiovascular disease or disorder, inflammatory disease or disorder, pulmonary disease or disorder, neurological disease or disorder, metabolic disease or disorder, dermatological disease or disorder, age-related disease or disorder, a premature aging disease or disorder, and a sleep disorder.

Particular conditions and diseases or disorders that are treated by the present methods, in various embodiments, include sarcopenia. Sarcopenia is characterized first by a muscle atrophy (a decrease in the size of the muscle), along with a reduction in muscle tissue “quality,” caused by such factors as replacement of muscle fibers with fat, an increase in fibrosis, changes in muscle metabolism, oxidative stress, and degeneration of the neuromuscular junction. Combined, these changes lead to progressive loss of muscle function and frailty.

In various embodiments, the methods and compositions of the present disclosure modulate (e.g., increase or decrease) levels of inflammation in a subject. “Inflammation” is a normal response to a variety of acute stresses on the body, including infection, fever and injury. Other types of inflammation include increased levels of pro-inflammatory cytokines found within tissues and systemically in plasma. Inflammation may be associated with infections, but it occurs in response to virtually any type of injury or threat, including physical trauma, cold, burns from radiation, heat or corrosive materials, chemical irritants, bacterial or viral pathogens, localized oxygen deprivation (ischemia) or reperfusion (sudden reinfusion of oxygen to ischemic tissue), and others. It includes the classic symptoms of redness, heat, swelling, and pain, and may be accompanied by decreased function of the inflamed organ or tissue.

Inflammation is a generalized reaction involving several effects that may tend to combat an injurious agent that may be present at the site where an injury or threat was detected, or it may tend to contain the injury or threat to its initial location, to keep it from spreading rapidly. Inflammation is a self-defensive reaction aimed at eliminating or neutralizing injurious stimuli, and restoring tissue integrity. Like peripheral inflammation, neuroinflammation can become a harmful process, and it is now widely accepted that it may contributes to the pathogenesis of many central nervous system disorders. CNS inflammation is commonly associated with some degree of tissue damage including, loss of myelin sheaths or loss of axons, and is a central theme in human patients with MS. The level of inflammation can be quantified by performing a simple blood test for a particular compound called C-reactive protein, or CRP.

In various embodiments, the methods of the present disclosure decrease levels of sterile chronic systemic inflammation in a subject. “Sterile chronic systemic inflammation,” is a characteristic of aging. Chronic inflammation causes damage over time to organ systems like the heart, brain, and kidneys, leading to disability or premature death. Blood vessels that supply these organs are vulnerable to inflammation, leading to vessel wall-thickening and narrowing of the blood passageway. Elevated CRP levels, measured over time, are an indicator of chronic inflammation in humans. Studies have shown that elevated levels of CRP correlate with an increased risk of heart attack and stroke. Aging is an intricate process that results from a combination of environmental, genetic, epigenetic, and stochastic factors. A chronic proinflammatory status is a pervasive feature of aging.

This chronic, low-grade, systemic inflammation occurring in the absence of overt infection (sterile inflammation) has been defined as “inflammaging” and represents a significant risk factor for morbidity and mortality in the elderly. Prattichizzo et al in (Inflammaging” as a Druggable Target: A Senescence-Associated Secretory Phenotype-Centered View of Type 2 Diabetes) Oxid Med Cell Longev. 2016 and Nasi et al in (Aging and inflammation in patients with HIV infection), Clin Exp Immunol. 2016 May 20, explore the connection between aging and inflammation.

In various embodiments, methods of the present disclosure improve, treat, diminish, attenuate, reduce, and/or delaying the onset of age-related diseases or disorders in a subject. The term “age-related disease or disorder” includes, but is not limited to, a disease or disorder in an adult such as cancer, a metabolic disease, cardiovascular disease, tobacco-related disease, or skin wrinkles. Cancer includes, but is not limited to, prostate cancer, colon cancer, lung cancer, squamous cell cancer of the head and neck, esophageal cancer, hepatocellular carcinoma, gastric cancer, pancreatic cancer, ovarian cancer, or breast cancer. Age-related or tobacco-related disease or disorder includes cardiovascular disease, cerebrovascular disease, peripheral vascular disease, Alzheimer's disease, osteoarthritis, cardiac diastolic dysfunction, benign prostatic hypertrophy, aortic aneurysm, or emphysema.

In various embodiments, methods of the present disclosure mediate rejuvenation in a subject. The term “rejuvenation” refers to the results of reducing or preventing the progress of aging and/or reducing or preventing the progress of an age-related disease or disorder. The term “rejuvenating” refers to a process of improving parameters of frailty index and/or other markers of aging cell phenotypes or markers of age-related disease or disorder states, e.g., improved muscle endurance or strength, improved glucose tolerance, decreased presence of systemic or local inflammatory cytokines, improved mitochondrial 1 0 function, and erasing epigenetic modifications participating in the cellular aging phenotype. In some embodiments, the loss or reduction of the expression at least one of the markers identified as having increased expression in adipose tissue macrophages (ATMs) from aged mice (Garg, S. K. et al. Crit Rev Immunol. 2014, 34(1):1-14.): CD11c, CD206, Mgl1, IL-6, TNF-alpha, Nos2, Ccr-7, IL-12, Arg1, Ccl-2, Ccr-1, Ccr-5, Ccr-9, Mcp-1, Cxcr-3, IL-1beta may also be considered a sign of rejuvenation.

### Frailty and Frailty Indices

The present disclosure provides methods and compositions for improving, treating, diminishing, attenuating, reducing, and/or delaying the onset of frailty in a subject by administering an AP-based agent (e.g., IAP) to the subject.

In some embodiments, frailty comprises an accumulation of deficiencies in major physiological functions, reduction of regeneration capabilities, impaired wound healing and increased risk of age-related diseases. For example, in some embodiments, frailty is associated with natural aging or accelerated aging. Frailty can be measured according to any number of indices or tests known to one of skill in the art. For example, one such index, the Physiological Frailty Index (PFI), includes measurement of one or more parameters selected from grip strength, systolic blood pressure, diastolic blood pressure, blood flow volume, number of blood neutrophils, percentage of blood neutrophils, number of blood monocytes, percentage of blood monocytes, number of lymphocytes, number of red blood cells, hemoglobin levels, hematocrit levels, mean corpuscular volume, mean corpuscular hemoglobin levels, mean corpuscular hemoglobin concentration and keratinocyte-derived cytokine levels. Deviation from a reference standard in any one individual is known as a deficit, and the overall average PFI score of the individual is a ratio of deficits to the total number of parameters measured.

Frailty can manifest as vulnerability to stressors and a reduced capacity to withstand stress. For example, the disclosure of Buchner and Wagner 1992 Clin Geriatr Med. 1992 February; 8(1):1-17 is hereby incorporated by reference in its entirety. Frailty can manifest as loss of complexity of homeostatic mechanisms (e.g., interconnectedness and/or feedback or feedforward). For example, the disclosure of Lipsitz 2002 J Gerontol A Biol Sci Med Sci. 2002 March; 57(3):B115-25.is hereby incorporated by reference in its entirety. Frailty can also manifest as disuse and/or a decrease in energy flow through an organism, as described in Bortz 2002, J Gerontol A Biol Sci Med Sci. 2002 May; 57(5):M283-8, which is hereby incorporated by reference in its entirety. Frailty can also manifest as homeostatic dysregulation, as described by Ferrucci 2005 J Gerontol. A Biol. Sci. Med. Sci. 60, 56, which is hereby incorporated by reference in its entirety.

There are several comprehensive approaches for quantitative assessment of aging-related accumulation of deficits and frailty in humans and animals. Individual organisms are heterogeneous in their health status and the rate of aging. To account for such heterogeneity, a Frailty Index (FI) has been introduced as a numerical score which is a ratio of the deficits present in a person to the total number of deficits considered in the study. Changes in the FI characterize the rate of individual aging. A similar approach has been applied to laboratory animals. Frailty index is considered as a reliable and broadly accepted measure of “biological age” and the degree of general health decline indicative of a reduction in the quality of life.

In certain aspects and embodiments, provided herein includes methods for improving and/or treating or reducing frailty and/or reducing frailty index in a patient. Frailty can be assessed in any of many methods known in the art. For example, frailty and methods to evaluate/index frailty are described in Hubbard, et al., Ageing, published electronically November, 2008 page 115-118; Cesari, et al., Age and Ageing, 43:10-12, 2014; and Mohler et al., Experimental Gerontology, 54:6-13, 2014, all of which are hereby incorporated by reference. Further, a clinical frailty index in aging mice is compared with frailty index data in humans, as described in Whitehead, et al., J Gerontol A Biol Sci Med Sci. 2014 June; 69(6): 621-632, which is hereby incorporated by reference. The researchers of the Whitehead paper established a simplified, noninvasive method to quantify frailty through clinical assessment of C57BL/6J mice (5-28 months) and compared the relationship between FI scores and age in mice and humans.

In various embodiments, a Frailty Index is calculated as described in U.S. Patent Application Publication No. 2015/0285823, which is incorporated herein by reference. For example, a description of the determination of the Frailty Index is provided. The Frailty Index was developed to assess a fit to frail range for the organisms of the same chronological age to address the notion that chronological age does not always reflect biologic age. Based on sixteen-item parameters (that include measurements of weight, grip strength, blood pressure, complete blood count, cytokine level analysis), FI is calculated as a ratio of the total number of deficits measured and are assigned a score of FI between 0 (no deficits=fit) and 1 (all deficits present=frail). Therefore, higher FI indicates poorer health of an organism. In this regard, a FI is provided as a useful tool for assessing a “fit” to “frail” range organisms of the same chronological age.

In certain embodiments, methods and compositions of the present disclosure improve, treat, diminish, attenuate, reduce, and/or delay the onset of frailty in a subject as measured according to the Physiological Frailty Index (PFI), as described in Antoch et al. Aging. 2017; 9: 1-12 (hereby incorporated by reference in its entirety). For example, PFI can be determined for an individual subject with reference to a young reference subject. For each subject, various parameters are measured. These parameters include non-invasive measurements, including age, body weight, grip strength, and diastolic blood pressure. Additional blood chemistry measurements may also be determined, including white blood cell count, neutrophil count, neutrophil percentage, lymphocyte percentage, monocyte percentage, eosinophil percentage, red blood cell count, hemoglobin levels, hematocrit levels, mean corpuscular volume, mean corpuscular hemoglobin levels, mean corpuscular hemoglobin concentration, platelet count, and mean platelet volume.

For each parameter mean value and standard deviation are calculated. Subjects differing in more than one standard deviation (STDEV) from mean value in any single parameter are excluded from the reference group. The value for each parameter measured for subjects of older ages is compared with the corresponding value for the reference group and assigned a score. Values that differ less than 1 STDEV are assigned the score of 0 (no deficit, within the range of the reference group). Values that are different for one STDEV are scored as 0.25 (minimal deficit). Values that differ from the corresponding values in the reference group by 2 STDEV are scored as 0.5 and those that differ by 3 STDEV are scored as 0.75. If the value is above 3 STDEV, it is scored as 1 (extreme deficit). The number of deficits the individual subject expressed is calculated as a ratio of the total number of parameters measured and is referred to as Physiological Frailty Index (PFI).

In some embodiments, methods of the present disclosure improve, treat, diminish, attenuate, reduce, and/or delay the onset of frailty in a subject, as measured by the PFI. For example, administering the AP-based agent to a subject to improve, treat, diminish, attenuate, reduce, and/or delay the onset of frailty can result in a reduced PFI score. In some embodiments, a subject's PFI score is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, a subject's PFI score is reduced by about 25%-75%, about 25%-50%, or about 50% to 75%. In further embodiments, a subject's PFI score is reduced to no greater than 0.9, 0.85, 0.8, 0.75, 0.7, 0.65, 0.6, 0.55, 0.5, 0.45, 0.4, 0.35, 0.3, 0.25, 0.2, 0.15, 0.1 or 0.5.

In some embodiments, frailty is quantified by the performance-based frailty index, which is a noninvasive clinical assessment and contains key features of the frailty index established for use in humans. This clinical assessment includes evaluation of the integument, the musculoskeletal system, the ocular and nasal systems, the digestive system, the urogenital system, the respiratory system, signs of discomfort, the body weight, and body surface temperature.

Further, frailty as an accumulation of deficits can be measured by the Rockwood frailty index, as described in Rockwood et al., J Gerontol A Biol Sci Med Sci. 2007 July; 62(7):722-727, which is incorporated by reference in its entirety. In embodiments, the present methods improve, treat, diminish, attenuate, reduce, and/or delay the onset of frailty as assessed by the Rockwood frailty index.

Frailty as a biologic syndrome of decreased reserve resulting from cumulative declines across multiple physiologic systems can be measured by the Fried frailty score, as described in Fried et al., J Gerontol A Biol Sci Med Sci. 2001 March; 56(3):M146-56, which is incorporated by reference in its entirety. The Fried frailty score comprises a Physical Frailty Phenotype (PFP), which measures various parameters, such as weight loss of more than 10 pounds; weakness as related to grip strength; self-reported exhaustion; 15 feet walking speed; and amount of physical activity in Kcals per week. The Fried frailty score incorporates scoring of 0 (not frail), 1-2 (intermediate frailty), and greater than or equal to 3 (frail). In various embodiments, methods of the present disclosure improve, treat, diminish, attenuate, reduce, and/or delay the onset of frailty in a subject, as measured by a Fried frailty score. For example, administering the AP-based agent to a subject improve, treat, diminish, attenuate, reduce, and/or delay the onset of frailty can result in a reduced Fried frailty score from 3 to 2, from 3 to 1, from 3 to 0, from 2 to 1, from 2 to 0 or from 1 to 0. Further, in some embodiments, administering the AP-based agent to a subject to improve, treat, diminish, attenuate, reduce, and/or delay the onset of frailty results in a lack of increase of a subject's Fried frailty score.

Frailty can also be measured by the FRAIL Scale, as described in Abellean Van Kan et al., J Am Med Dir Assoc. 2008 February; 9(2):71-2. doi: 10.1016/j.jamda.2007.11.005, which is incorporated by reference in its entirety. The parameters measured in the FRAIL Scale include feelings of persistent fatigue; resistance (ability to climb a single flight of stairs); ambulation (ability to walk one block); more than five illnesses; and more than 5% loss of weight. The FRAIL Scale incorporates scoring of 0 (not frail), 1-2 (intermediate frailty), and greater than or equal to 3 (frail). In various embodiments, methods of the present disclosure reduce or improve frailty in a subject, as measured by a FRAIL Scale score. For example, administering the AP-based agent to a subject in order to reduce or improve frailty can result in a reduced FRAIL Scale score from 3 to 2, from 3 to 1, from 3 to 0, from 2 to 1, from 2 to 0 or from 1 to 0. Further, in some embodiments, administering the AP-based agent to a subject to improve, treat, diminish, attenuate, reduce, and/or delay the onset of frailty results in a lack of increase of a subject's FRAIL Scale score.

In some embodiments the methods and compositions as provided herein improve (or reduce) the frailty index, or delay or slow a decline in frailty using at least one accepted measure of frailty. In some embodiments the methods as provided herein improve (or reduce) frailty index, or delay or slow a decline in frailty using at least one accepted measure of frailty selected from the Frailty Index (FI), the Physiological Frailty Index (PFI), Fried frailty score, Rockwood frailty index, FRAIL Scale and the modified frailty index.

In some embodiments, the frailty comprises low lean mass, weakness, exhaustion, low energy expenditure and/or slow walking speed. In embodiments, the present methods reduce or delay the onset or development of one or more of low lean mass, weakness, exhaustion, low energy expenditure and/or slow walking speed.

### Age-Related Changes to Gastrointestinal Microbiota

The present disclosure further contemplates embodiments providing methods for improving, treating, diminishing, attenuating, reducing, slowing the progression of, delaying the onset of, and/or reversing age-related changes of gastrointestinal microbiota phylum diversity by administering an AP-based agent to a subject in need thereof. Specifically, the disclosure provides methods for reversing and or delaying age-associated changes in commensal bacterial populations in the gastrointestinal microbiome. For example, gastrointestinal microbiota phyla that may be affected by age-related changes include, but are not limited to, Proteobacteria, Actinobacteria, Epsilonbactareota, Deferribacteres, Tenericutes, and Verrucomicrobia. In some embodiments, age-related changes of gastrointestinal microbiota phylum diversity result in a decrease in the abundance of the microbiota phylum selected from one or more of Proteobacteria, Actinobacteria, Epsilonbactareota, and Deferribacteres. In some embodiments, age-related changes of gastrointestinal microbiota phylum diversity results in an increase in the abundance of the microbiota phylum selected from Tenericutes and Verrucomicrobia.

For example, in various embodiments, age-related changes of gastrointestinal microbiota phylum diversity result in at least a 10%, 15%, 20%, 25%, 30%, 35%, 40%, 45%, 50%, 55%, 60%, 65%, 70%, 75%, 80%, 85%, 90%, or 95% abundance decrease in one or more of Proteobacteria, Actinobacteria, Epsilonbactareota, and Deferribacteres before treatment. In some embodiments, age-related changes of gastrointestinal microbiota phylum diversity result in at least a 10%, 15%, 20%, 25%, 30%, 35%, 40%, 45%, 50%, 55%, 60%, 65%, 70%, 75%, 80%, 85%, 90%, or 95% abundance increase in one or more of Tenericutes and Verrucomicrobia before treatment.

In various embodiments, administering the AP-based agent to the subject in need thereof results in gastrointestinal microbiota phylum diversity similar to the phylum diversity exhibited by a patient not having an age-related change. For example, in embodiments, age-related changes of gastrointestinal microbiota phylum diversity are reversed and/or normalized by at least a 10%, 15%, 20%, 25%, 30%, 35%, 40%, 45%, 50%, 55%, 60%, 65%, 70%, 75%, 80%, 85%, 90%, or 95% abundance increase in one or more of Proteobacteria, Actinobacteria, Epsilonbactareota, and Deferribacteres after treatment. In some embodiments, age-related changes of gastrointestinal microbiota phylum diversity are reversed and/or normalized by at least a 10%, 15%, 20%, 25%, 30%, 35%, 40%, 45%, 50%, 55%, 60%, 65%, 70%, 75%, 80%, 85%, 90%, or 95% abundance decrease in one or more of Tenericutes and Verrucomicrobia after treatment.

In various embodiments, age-associated changes to the gastrointestinal microbiota are manifested in changes to fecal microbial composition. In some embodiments, the composition and/or diversity of the gastrointestinal microflora is measured by 16S rRNA sequencing and analysis of fecal samples at various time points. In further embodiments, gastrointestinal microbiota diversity is measured in observed taxonomic units (OTUs) and/or according to the Shannon diversity index. In various embodiments, the Principal Component Analysis (PCA) assesses the relative abundance of microbiota phyla.

The Shannon Diversity Index refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pi is the proportion of R made up of the i^{th} species. Higher values indicate diverse and equally distributed communities, and a value of 0 indicates only one species is present in a given community. For further reference, see Reese and Dunn, Am. Soc'y Microbio. July/August 2018 Volume 9 Issue 4 e01294-18.

In certain embodiments, the present methods and compositions provide for restoration or maintenance of sufficient bacterial richness and diversity in the gut microbiota to offset or delay the onset of deleterious effects of aging. In various embodiments, the present methods and compositions provide functional redundancy, adaptability and/or systematic robustness against age-related challenges.

In certain embodiments, the present methods and compositions provide for an increased Shannon diversity index of the gastrointestinal microbiota of a subject being administered the present AP-based agent.

In various embodiments, the present methods and compositions provide for screening of a subject's gastrointestinal microbiota as described herein and, if reflective of age-related changes, administering the present AP-based agents to reverse or delay such changes.

### Accelerated Aging (Progeroid Syndromes)

In some embodiments, the present disclosure provides methods and compositions for reducing accelerated aging in a subject. For instance, in some embodiments, the present disclosure relates to the administration of an AP-based agent (e.g., IAP) to a subject or patient to reduce accelerated aging associated with progeroid syndromes.

In various embodiments, methods of the present disclosure include improving, treating, diminishing, attenuating, reducing, slowing the progression of, and/or delaying the onset of premature or accelerated aging. In some embodiments, accelerated aging is a symptom of any one of the progeroid syndromes, including, but not limited to, Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), or restrictive dermopathy (RD). Subjects having one of these diseases or disorders typically has reduced longevity (i.e., lifespan).

### Lifespan

In some embodiments, the present disclosure provides methods for increasing a subject's longevity or lifespan. For instance, in some embodiments, the present disclosure relates to the administration of an AP-based agent (e.g., IAP) to a patient to increase longevity or lifespan.

For example, the present methods and compositions can increase a subject's longevity or lifespan by at least about 1 month, at least about 3 months, at least about 6 months, at least about 9 months, at least about 1 year, at least about 5, at least about 10, at least about 15, at least about 20, or at least about 25 years, as compared to a subject that is not administered the AP-based agent described herein and/or as compared to a life expectancy calculation, as described herein. Further, various embodiments of the present methods and compositions increase cellular autophagy in a subject.

In various embodiments, an increase in longevity or lifespan is assessed relative to a comparable population. For example, an increase in longevity or lifespan is assessed relative to a cohort—e.g. cohort LEB, the mean length of life of an actual birth cohort (all individuals born a given year) or a period—e.g. period LEB, the mean length of life of a hypothetical cohort assumed to be exposed, from birth through death, to the mortality rates observed at a given year. Such assessments can be made relative to various reports on lifespan and/or longevity in the art (e.g. World Health Organization (WHO)'s Health Status Statistics: Mortality). In some embodiments, the present methods provide for increased longevity or lifespan than what is expected relative to comparable populations. In some embodiments, the present methods provide for increased longevity or lifespan than what is expected relative to various reports on lifespan and/or longevity in the art (e.g. World Health Organization (WHO)'s Health Status Statistics: Mortality).

In further embodiments, an increase in longevity or lifespan is assessed with reference to one or more actuarial life tables, e.g., Life Tables for the United States Social Security Area 1900-2100 (Actuarial Study No. 120, Bell and Miller). In some embodiments, the present methods provide for increased longevity or lifespan than what is expected relative to one or more actuarial life tables.

### Subjects

The methods provided herein can be used with a patient that is a mammal, including humans and non-human mammals. Non-human mammals treated using the present methods include domesticated animals (e.g., canine, feline, primates, murine, rodentia, and lagomorpha) and agricultural animals (e.g., bovine, equine, ovine, and porcine). In various examples, the individual to whom a compound or composition is administered is an individual who is at risk for, is suspected of having or has been diagnosed with an age-related disease or disorder.

In various embodiments of the present disclosure, the patient is a young human, a middle-aged human, or an elderly human. For example, in some embodiments, the patient is between about 18 and about 35 years, or between about 18 and about 30 years, or between about 18 and about 25 years, or between about 18 and about 20 years. In some embodiments, the patient is between about 36 and about 55 years, or between about 40 and about 55 years, or between about 45 and about 55 years, or between about 36 and about 50 years, or between about 36 and about 45 years, or between about 36 and about 40 years, or between about 40 and about 50 years old, or between about 45 and about 55 years old. In some embodiments, the patient is between about 56 and about 85 years, or between about 60 and about 85 years, or about 65 and about 85 years, or between about 70 and about 85 years, or between about 75 and about 85 years, or between 80 and about 85 years, or between 56 and about 80 years, or between 56 and about 75 years, or between 56 and about 70 years, or between 56 and about 65 years, or between 56 and about 60 years, or between about 60 years and about 80 years, or about 65 years and about 75 years.

In some embodiments, the patient is about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85years old. In some embodiments, the patient is at least 55 years old.

A person of skill in the art will contemplate that age ranges with respect to “young,” “middle-aged,” and “elderly” definitions can vary based on geographic region, among other factors. Petry, Gerontologist 2002 February; 42(1):92-9, describes age-related definitions and is hereby incorporated by reference in its entirety.

In embodiments, the biological sex of the patient is male or female. In embodiments, the biological sex of the patient is male. In embodiments, the biological sex of the patient is female.

In embodiments, the patient is middle aged (e.g. between about 36 and about 55 years, or between about 40 and about 55 years, or between about 45 and about 55 years, or between about 36 and about 50 years, or between about 36 and about 45 years, or between about 36 and about 40 years, or between about 40 and about 50 years old, or between about 45 and about 55 years old). In some embodiments, the present methods, e.g., as applicable to a middle-aged male patient, improve, treat, diminish, attenuate, reduce, and/or delay the onset of the severity of one or more frailties and age-related diseases or disorders.

In various embodiments of the present disclosure, the subject is a human patient. In some embodiments, the patient is a middle-aged human. For example, in some embodiments, the patient is between about 35 and 55 years old.

In various embodiments, the patient is an elderly and/or geriatric human. For example, in some embodiments, the patient is between about 56 and about 85 years old. In some embodiments, an elderly patient is equal to or older than about 65 years old.

In some embodiments of the methods provided herein, the patient is a mammal. In some embodiments of the methods provided herein, the patient is a human.

### AP-Based Agents and Compositions

The present disclosure is directed, in part, to pharmaceutical compositions, formulations, and uses of one or more alkaline phosphatases. Alkaline phosphatases are dimeric metalloenzymes that catalyze the hydrolysis of phosphate esters and dephosphorylate a variety of target substrates at physiological and higher pHs. Illustrative APs that may be utilized in the present disclosure include, but are not limited to, intestinal alkaline phosphatase (IAP; e.g., calf IAP or bovine IAP, chicken IAP, goat IAP), placental alkaline phosphatase (PLAP), placental-like alkaline phosphatase, germ cell alkaline phosphatase (GCAP), tissue non-specific alkaline phosphatase (TNAP; which is primarily found in the liver, kidney, and bone), bone alkaline phosphatase, liver alkaline phosphatase, kidney alkaline phosphatase, bacterial alkaline phosphatase, fungal alkaline phosphatase, shrimp alkaline phosphatase, modified IAP, recombinant IAP, or any polypeptide comprising alkaline phosphatase activity.

In various embodiments, the present disclosure contemplates the use of mammalian alkaline phosphatases including, but are not limited to, intestinal alkaline phosphatase (IAP), placental alkaline phosphatase (PLAP), germ cell alkaline phosphatase (GCAP), and the tissue non-specific alkaline phosphatase (TNAP).

### Intestinal Alkaline Phosphatase (IAP)

In some embodiments, the alkaline phosphatase is IAP. IAP is produced in the proximal small intestine and is bound to the enterocytes via a glycosyl phosphatidylinositol (GPI) anchor. Some IAP is released into the intestinal lumen in conjunction with vesicles shed by the cells and as soluble protein stripped from the cells via phospholipases. The enzyme then traverses the small and large intestine such that some active enzyme can be detected in the feces. In an embodiment, the IAP is human IAP (hIAP). In an embodiment, the IAP is calf IAP (cIAP), also known as bovine IAP (bIAP). There are multiple isozymes of bIAP, for example, with bIAP II and IV having higher specific activity than bIAP I. In an embodiment, the IAP is any one of the cIAP or bIAP isozymes (e.g., bIAP I, II, and IV). In an embodiment, the IAP is bIAP II. In another embodiment, the IAP is bIAP IV.

In various embodiments, the IAP of the present disclosure has greater or equal specific enzymatic activity than commercially-available APs, e.g., calf IAP (cIAP). IAP variants

Also included within the definition of IAPs are IAP variants. An IAP variant has at least one or more amino acid modifications, generally amino acid substitutions, as compared to the parental wild-type sequence. In some embodiments, an IAP of the present disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, IAP variants retain most or all of their biochemical activity, measured as described herein.

### GPI Anchored Proteins

Mammalian alkaline phosphatases are glycosylphosphatidylinositol (GPI)-anchored proteins. They have signal peptides and are translated into the secretory pathway. Once in the endoplasmic reticulum (ER), the proteins are glycosylated and folded. There are two disulfide bonds as well as a single free cysteine that is apparently not accessible on the surface. In the late ER, the carboxy terminus is removed and the GPI anchor is appended. GPI anchoring is therefore a process that occurs at the carboxy terminus of the alkaline phosphatase. The inclusion of stop codons at the anchor site enables secretion of biologically active protein (presumably the homodimer). While there is no consensus sequence, the carboxy terminus includes three amino acids, termed omega, omega+1, and omega+2 which are followed by a short stretch of hydrophilic amino acids and then a stretch of hydrophobic amino acids. Without wishing to be bound by theory, it is believed that the hydrophobicity is critical for embedding the carboxy terminus in the ER membrane. There, an enzymatic reaction replaces the carboxy terminus with the GPI anchor.

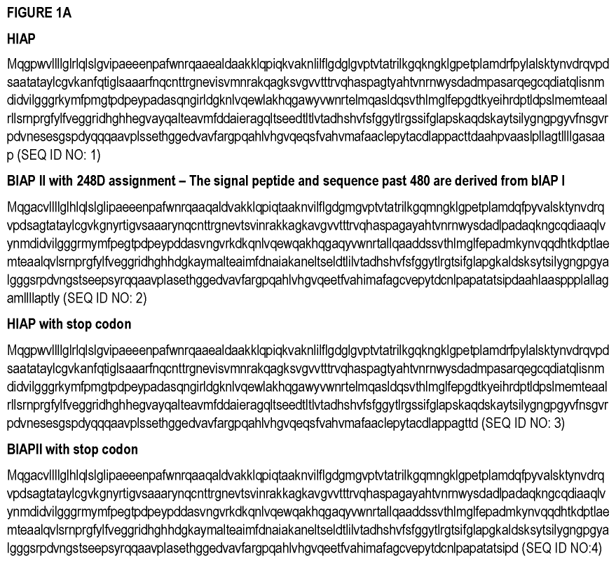

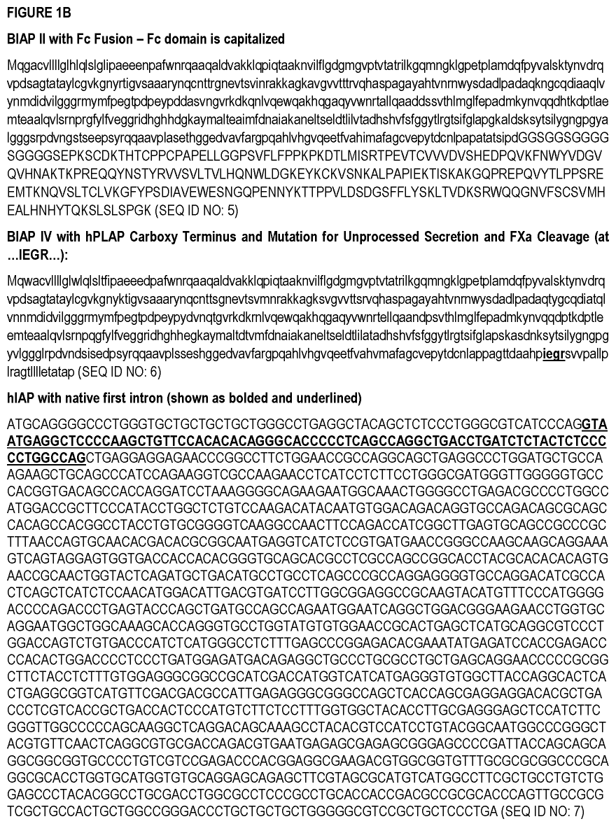

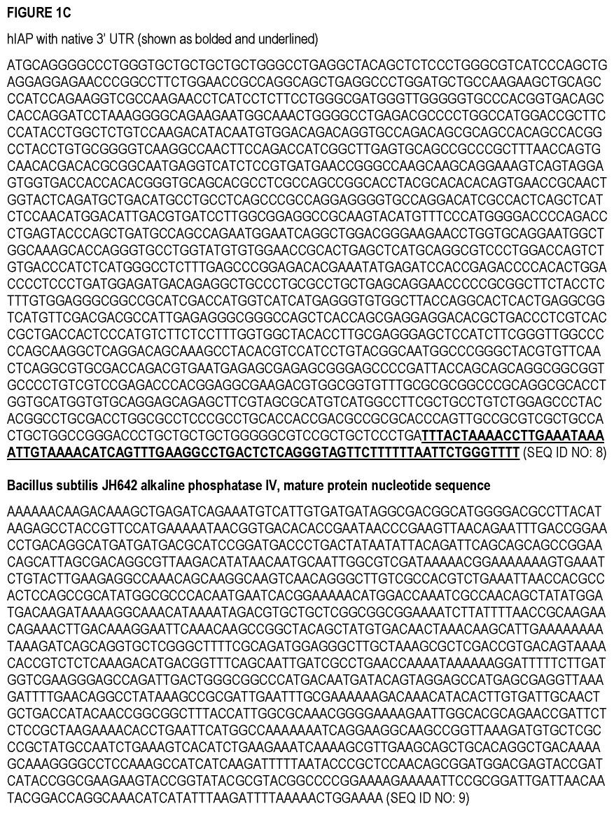

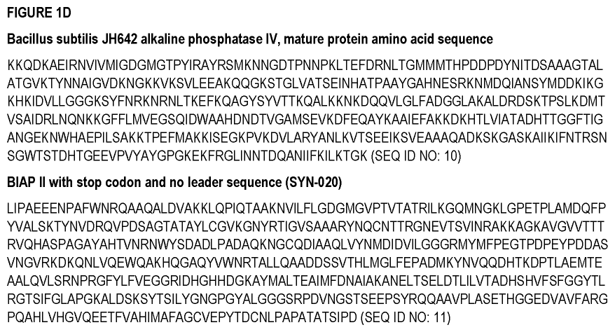

In other embodiments, the IAP of the disclosure is a secreted protein; that is, in some embodiments, the IAP is not GPI anchored, leading to secretion rather than intracellular retention. This can be accomplished in several ways. In some embodiments, the IAP may lack the GPI anchor site, e.g. have the DAAH site removed, leading to secretion. Alternatively, this can be accomplished in some embodiments, the IAP comprises a stop codon that is inserted immediately before the GPI anchor site. In an embodiment, the IAP comprises a stop codon after the aspartate in the DAAH consensus site (e.g., at amino acid 503 of hIAP and bIAP IV or amino acid 506 of bIAP II). FIG. 1A depicts HIAP with a stop codon (SEQ ID NO: 3) and bIAP II with a stop codon (SEQ ID NO: 4).

### Human IAP

In various embodiments, the IAP is human IAP (hIAP). In some embodiments, the IAP is hIAP comprising the amino acid sequence of SEQ ID NO: 1 as depicted in FIG. 1A or a variant as described herein, as long as the hIAP variant retains at least 75, 80, 85, 90, 95, 96, 97, 98, 99 or 100% of the phosphatase activity as compared to the wild type enzyme using an assay as outlined herein.

Included within the definition of hIAP are amino acid modifications, with amino acid substitutions finding particular use in the present disclosure. For example, without wishing to be bound by theory, it is believed that a cysteine at the carboxy terminus of the AP-based agent (e.g., at position 500 of SEQ ID NO: 1) may interfere with protein folding. Accordingly, in some embodiments, the AP-based agent includes a mutation of the cysteine (e.g., at position 500 of SEQ ID NO: 1). In some embodiments, the cysteine is replaced with any amino acid, although glycine finds particular use in some embodiments. Furthermore, the C-terminal cysteine can also be deleted.

As will be appreciated by those in the art, additional amino acid modifications can be made in hIAP as discussed herein. For example, in some embodiments, a stop codon may be inserted after the aspartate in the DAAH consensus site (e.g., at amino acid 503 of hIAP). FIG. 1A depicts hIAP with an inserted stop codon (SEQ ID NO: 3).

### Fusion Proteins

In various embodiments, the present disclosure provides for chimeric proteins. In some embodiments, the present disclosure provides for chimeric fusion proteins. For example, in various embodiments, the present disclosure provides an isolated or recombinant alkaline phosphatase comprising a crown domain and a catalytic domain, wherein said crown domain and said catalytic domain are obtained from different alkaline phosphatases (e.g., human and bovine alkaline phosphatases). In other embodiments, the alkaline phosphatases are both human APs. In certain embodiments, the present disclosure provides for recombinant fusion proteins comprising human IAP and a domains of human placental alkaline phosphatases. In certain embodiments, the present disclosure provides for chimeric hIAP-placenta fusion proteins.

In various embodiments, the AP-based agent of the disclosure is a fusion protein. In some embodiments, the AP-based agent comprises an alkaline phosphatase fused to a protein domain that replaces the GPI anchor sequence. In some embodiments, the alkaline phosphatase is fused to a protein domain that promotes protein folding and/or protein purification and/or protein dimerization and/or protein stability. In various embodiments, the AP-based agent fusion protein has an extended serum half-life.

In an embodiment, the alkaline phosphatase is fused to an immunoglobulin Fc domain and/or hinge region. In various embodiments, the immunoglobulin Fc domain and/or hinge region is derived from the Fc domain and/or hinge region of an antibody (e.g., of IgG, IgA, IgD, and IgE, inclusive of subclasses (e.g. IgG1, IgG2, IgG3, and IgG4, and IgA1 and IgA2)). In an embodiment, the AP-based agent of the disclosure comprises an alkaline phosphatase fused to the hinge region and/or Fc domain of IgG.

In various embodiments, the AP-based agent of the disclosure is a pro-enzyme. In an embodiment, the activity of the proenzyme is suppressed by a carboxy terminus. In an embodiment, protease removal of the carboxy terminus reactivates the enzymatic activity of the alkaline phosphatase. In an embodiment, the pro-enzyme is more efficiently secreted than the enzyme without the carboxy terminus.

In some embodiments, for generation of the pro-enzyme, the native carboxy terminus of the alkaline phosphatase is replaced with the analogous sequence from hPLAP. In some embodiments, a mutation is made in the hydrophobic carboxy tail to promote protein secretion without cleavage of the carboxy terminus. In an illustrative embodiment, a single point mutation such as a substitution of leucine with e.g., arginine is generated in the hydrophobic carboxy terminus (e.g., allpllagtl is changed to, e.g., allplragtl) to result in secretion of the enzyme without removal of the carboxy terminus.

### Bovine IAPs

In some embodiments, the IAP is bovine IAP (bIAP).

In various embodiments, the IAP is bovine IAP II (bIAP II) or a variant as described herein, as long as the bIAP variant retains at least 75, 80, 85, 90, 95, 96, 97, 98, 99 or 100% of the phosphatase activity using an assay as outlined herein. In an embodiment, the bIAP II comprises the signal peptide and carboxy terminus of bIAP I. In an embodiment, the bIAP II comprises an aspartate at position 248 (similar to bIAP IV). In an embodiment, the bIAP II comprises the amino acid sequence of SEQ ID NO: 2. FIG. 1A depicts BIAP II with 248D assignment — SEQ ID NO: 2. The signal peptide and sequence past 480 are derived from bIAP I.

Also included within the definition of bIAP II are amino acid variants as described herein. For example, in some embodiments, a stop codon may be inserted after the aspartate in the DAAH consensus site (e.g., at amino acid 506 of bIAP II). FIG. 1A depicts bIAP II with an inserted stop codon (SEQ ID NO: 4).

In various embodiments, the bIAP II comprises the amino acid sequence of SEQ ID NO: 11.

BIAP II with stop codon and no leader sequence
(SYN-020) (SEQ ID NO: 11):
LIPAEEENPAFWNRQAAQALDVAKKLQPIQTAAKNVILFLGDGMGVPTVT
ATRILKGQMNGKLGPETPLAMDQFPYVALSKTYNVDRQVPDSAGTATAYL
CGVKGNYRTIGVSAAARYNQCNTTRGNEVTSVINRAKKAGKAVGVVTTTR
VQHASPAGAYAHTVNRNVVYSDADLPADAQKNGCQDIAAQLVYNMDIDVI
LGGGRMYMFPEGTPDPEYPDDASVNGVRKDKQNLVQEWQAKHQGAQYVWN
RTALLQAADDSSVTHLMGLFEPADMKYNVQQDHTKDPTLAEMTEAALQVL
SRNPRGFYLFVEGGRIDHGHEIDGKAYMALTEAIMFDNAIAKANELTSEL
DTLILVTADHSHVFSFGGYTLRGTSIFGLAPGKALDSKSYTSILYGNGPG
YALGGGSRPDVNGSTSEEPSYRQQAAVPLASETHGGEDVAVFARGPQAHL
VHGVQEETFVAHIMAFAGCVEPYTDCNLPAPATATSIPD.

### Expression Variants

In various embodiments, the IAP of the disclosure is efficiently expressed and secreted from a host cell. In an embodiment, the IAP of the disclosure is efficiently transcribed in a host cell. In another embodiment, the IAP exhibits enhanced RNA stability and/or transport in a host cell. In another embodiment, the IAP is efficiently translated in a host cell. In another embodiment, the IAP exhibits enhanced protein stability.

In various embodiments, the IAPs are efficiently expressed in a host cell. In an embodiment, the Kozak sequence of the DNA construct encoding the AP-based agent is optimized. The Kozak sequence is the nucleotide sequence flanking the ATG start codon that instructs the ribosome to start translation. There is flexibility in the design of a Kozak sequence, but one canonical sequence is GCCGCCACCATGG (SEQ ID NO:12). The purine in the −3 position and the G in the +4 position are the most important bases for translation initiation. For hIAP, bIAP II, and bIAP IV, the second amino acid, that is, the one after the initiator methionine, is glutamine. Codons for glutamine all have a C in the first position. Thus, their Kozak sequences all have an ATGC sequence. Accordingly, in various embodiments, the ATGC sequence is changed to ATGG. This can be achieved by changing the second amino acid to a glycine, alanine, valine, aspartate, or glutamic acid, all of whose codons have a G in the first position. These amino acids may be compatible with signal peptide function. In alternative embodiments, the entire signal peptide is substituted for peptide having a canonical Kozak sequence and is derived from a highly expressed protein such as an immunoglobulin.

In various embodiments, the signal peptide of the IAP may be deleted and/or substituted. For example, the signal peptide may be deleted, mutated, and/or substituted (e.g., with another signal peptide) to ensure optimal protein expression.

In some embodiments, the DNA construct encoding the IAP of the disclosure comprises untranslated DNA sequences. Such sequences include an intron, which may be heterologous to the IAP protein or native to the IAP protein including the native first and/or second intron and/or a native 3′ UTR. Without wishing to be bound by theory, it is believed that include of these sequences enhance protein expression by stabilizing the mRNA. Accordingly, in various embodiments, the DNA construct encoding the IAP of the disclosure comprises the 5′UTR and/or the 3′UTR. Provided in FIG. 1A-D are illustrative IAP DNA sequences with a first intron and a 3′UTR, including hIAP with native first intron (shown as bolded and underlined)—SEQ ID NO: 7; and MAP with native 3′ UTR (shown as bolded and underlined)—SEQ ID NO: 8.

In various embodiments, the IAP of the disclosure comprises a nucleotide sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein, or with a codon-optimized version thereof.

In various embodiments, the IAP of the disclosure may comprise an amino acid sequence having one or more amino acid mutations relative to any of the protein sequences described herein. In some embodiments, the one or more amino acid mutations may be independently selected from substitutions, insertions, deletions, and truncations.

In various embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ϵ-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N a-methyl amino acids, and amino acid analogs in general).

Mutations may be made to the IAP of the disclosure to select for agents with desired characteristics. For examples, mutations may be made to generate IAPs with enhanced catalytic activity or protein stability. In various embodiments, directed evolution may be utilized to generate IAPs of the disclosure. For example, error-prone PCR and DNA shuffling may be used to identify mutations in the bacterial alkaline phosphatases that confer enhanced activity.

### Administration and Dosages

It will be appreciated that the actual dose of the AP-based agent (e.g., IAP) administered according to the present disclosure will vary according to the particular compound, the particular dosage form, and the mode of administration. Many factors that may modify the action of the AP-based agent (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In various embodiments, the present disclosure contemplates that the AP-based agent is administered as a supplement, for example as a food additive. In further embodiments, the AP-based agent is chronically administered to the subject, e.g., for at least one year, or at least two years, or at least three years, or at least four years, or at least five years, or for the entirety of the subject's lifespan.

Individual doses of the AP-based agent (e.g., IAP) can be administered in unit dosage forms (e.g., tablets or capsules) containing, for example, from about 0.01 mg to about 1,000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, or from about 0.1 mg to about 1 mg active ingredient per unit dosage for. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg of the AP-based agent, inclusive of all values and ranges therebetween.

In one embodiment, the AP-based agent (e.g., IAP) is administered at an amount of from about 0.01 mg to about 1,000 mg daily, about 0.01 mg to about 900 mg daily, about 0.01 mg to about 800 mg daily, about 0.01 mg to about 700 mg daily, about 0.01 mg to about 600 mg daily, about 0.01 mg to about 500 mg daily, about 0.01 mg to about 400 mg daily, about 0.01 mg to about 300 mg daily, about 0.01 mg to about 200 mg daily, about 0.01 mg to about 100 mg daily, an amount of from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the IAP is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg, inclusive of all values and ranges therebetween.

In some embodiments, a suitable dosage of the AP-based agent (e.g., IAP) is in a range of about 0.01 mg/kg to about 100 mg/kg of body weight of the subject, about 0.01 mg/kg to about 90 mg/kg of body weight of the subject, about 0.01 mg/kg to about 80 mg/kg of body weight of the subject, about 0.01 mg/kg to about 70 mg/kg of body weight of the subject, about 0.01 mg/kg to about 60 mg/kg of body weight of the subject, about 0.01 mg/kg to about 50 mg/kg of body weight of the subject, about 0.01 mg/kg to about 40 mg/kg of body weight of the subject, about 0.01 mg/kg to about 30 mg/kg of body weight of the subject, about 0.01 mg/kg to about 20 mg/kg of body weight of the subject, about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other embodiments, a suitable dosage of the AP-based agent is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the disclosure, the AP-based agent (e.g., IAP) may be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

In certain embodiments, an AP-based agent (e.g., IAP) in accordance with the methods provided herein is administered enterally or parenterally, for example, orally, subcutaneously (s.c.), intravenously (i.v.), intramuscularly (i.m.), intranasally or topically. Administration of an AP-based agent (e.g., IAP) described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses.

Various modes of administration of an AP-based agent (e.g., IAP) are contemplated herein. In some embodiments, an AP-based agent (e.g., IAP) is administered enterally. In such embodiments, the AP-based agent (e.g., IAP) is administered orally. For example, in embodiments, the AP-based agent is administered orally via a tablet or an encapsulated pellet, and in some embodiments, the tablet or pellet is enterically coated. Enteric coating can protect the AP-based agent from degradation in stomach fluid. In further embodiments, the tablet or pellet is formulated to release the AP-based agent in one or more of the proximal small intestine, the distal small intestine, and the colon. In some embodiments, the AP-based agent is administered as a food supplement and/or additive.

In one embodiment, an AP-based agent (e.g., IAP) is administered parenterally. In some embodiments, an AP-based agent is administered by injection, e.g. intramuscular injection. In some embodiments, administration is accomplished using a kit as described herein (e.g. via a unit dose form, e.g. a pre-loaded (a.k.a. pre-dosed or pre-filled) syringe or a pen needle injector (injection pen)).

### Kits

The disclosure provides kits that can simplify the administration of any agent described herein. An illustrative kit of the disclosure comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the disclosure and an effective amount of another composition, such those described herein.

### Definitions

With respect to the agents and compositions described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit,” “reduce,” and “remove” as used herein refer to any inhibition, reduction, decrease, suppression, downregulation, or prevention in expression, activity or symptom and include partial or complete inhibition of activity or symptom. Partial inhibition can imply a level of expression, activity or symptom that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression, activity or symptom. The terms “eliminate” or “eradicate” indicate a complete reduction of activity or symptom.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those that may not be discernible by the patient. In yet another embodiment, “treating” or “treatment” refers to modulating the disease or disorder, either physically (e.g., stabilization of a discernible symptom) or physiologically (e.g., stabilization of a physical parameter), or both. In yet another embodiment, “treating” or “treatment” refers to improving, diminishing, attenuating, reducing, slowing the progression of, and/or delaying the onset or development or progression of the disease or disorder.

As used herein, the term “abnormal” refers to an activity or feature that differs from a normal activity or feature. As used herein, the term “abnormal activity” refers to an activity that differs from the activity of the wild-type or native gene or protein, or which differs from the activity of the gene or protein in a healthy subject. The abnormal activity can be stronger or weaker than the normal activity. In one embodiment, the “abnormal activity” includes the abnormal (either over- or under-) production of mRNA transcribed from a gene. In another embodiment, the “abnormal activity” includes the abnormal (either over- or under-) production of polypeptide from a gene. In another embodiment, the abnormal activity refers to a level of a mRNA or polypeptide that is different from a normal level of the mRNA or polypeptide by about 15%, about 25%, about 35%, about 50%, about 65%, about 85%, about 100% or greater. In some embodiments, the abnormal level of the mRNA or polypeptide can be either higher or lower than the normal level of the mRNA or polypeptide. Yet in another embodiment, the abnormal activity refers to functional activity of a protein that is different from a normal activity of the wild-type protein. In some embodiments, the abnormal activity can be stronger or weaker than the normal activity. In some embodiments, the abnormal activity is due to the mutations in the corresponding gene, and the mutations can be in the coding region of the gene or non-coding regions such as transcriptional promoter regions. The mutations can be substitutions, deletions, insertions.

“Therapeutically effective amount” as used herein means the amount of a compound or composition (such as described herein) that causes at least one desirable change in a cell, population of cells, tissue, individual, patient or the like. In some embodiments a therapeutically effective amount as used herein means the amount of a compound or composition (such as described herein) that inhibits or provides a clinically significant change in a disease or disorder or condition (e.g., reduce by at least about 30 percent, at least about 50 percent, or at least about 90 percent) or in one or more features of a disease or disorder or condition described herein.

### EXAMPLES

The present disclosure will be further described in the following examples, which do not limit the scope of any disclosure or disclosures described in the claims.

### Example 1: Age-dependent Decline of IAP Activity Associated with Age-Related Physiological Alterations

This example first describes the effects of an age-dependent decline of IAP activity on the physiological alterations of increased gastrointestinal permeability and systemic inflammation. Second, this example establishes that a lack of IAP is associated with severe aging-related liver inflammation, steatosis, and increased proinflammatory characteristics (e.g., increased cytokines in portal serum).

### IAP Associated with Human Aging and Mouse Model

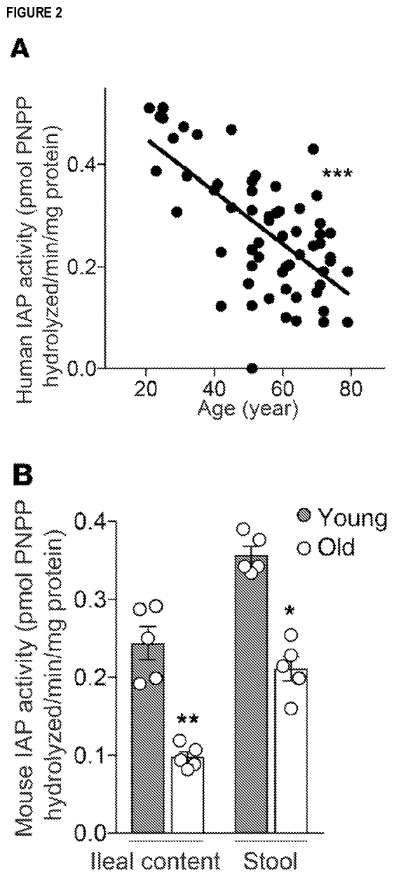

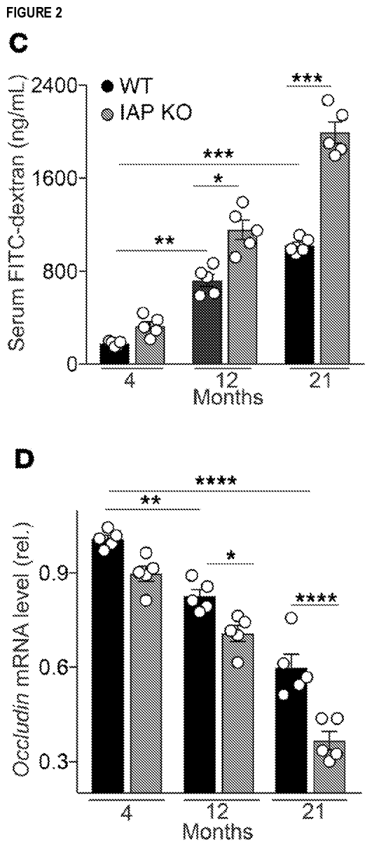

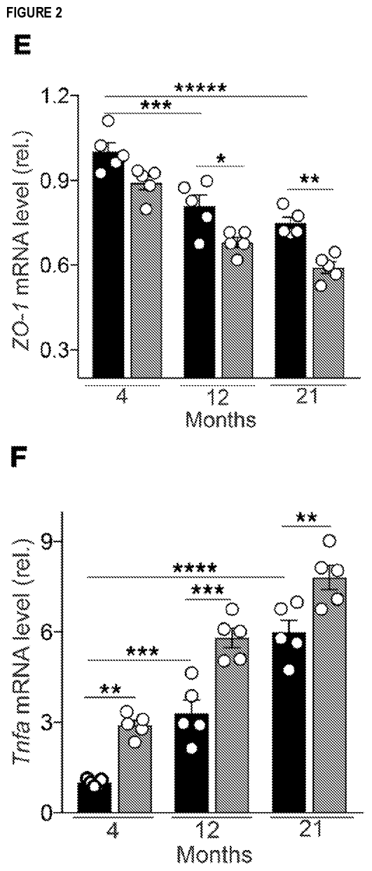

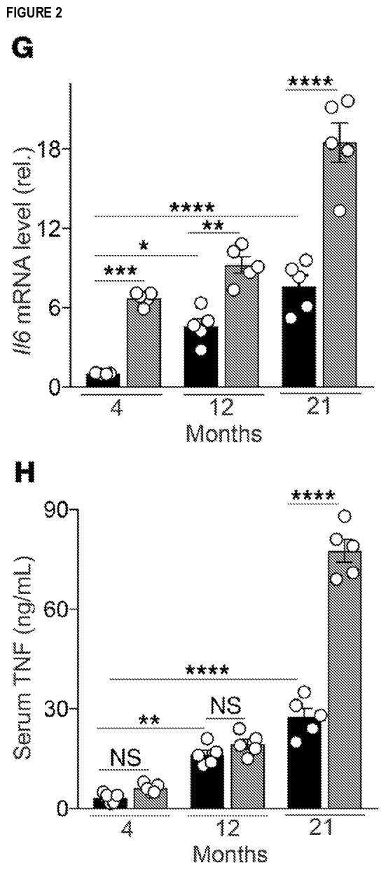

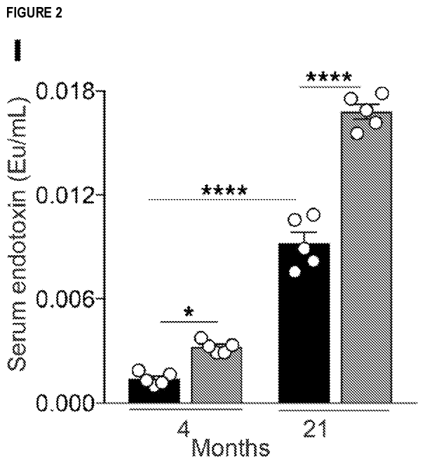

To determine if IAP plays a role in human aging, IAP activity was first tested in ileal contents from 60 stoma patients of different ages. Ileal fluid samples were collected from patients with an ileostomy and seen in the surgical clinic at the MGH. Demographics and clinical characteristics were obtained from the medical records. FIG. 2A depicts a significant decline of IAP activity with increasing age. To confirm the validity of the mouse model, IAP activity was similarly measured in mice and was found to decrease with age in mice. IAP activity was measured in stool and ileal content of young (4-month-old) and old (21-month-old) WT C57BL/6J mice by IAP assay. FIG. 2B shows that IAP activity was significantly lower in both the ileal fluid and the stool of old mice.

Gastrointestinal permeability was also measured in IAP-KO and WT mice of different age groups. In each set of two histograms of FIGS. 2C-I, the left bar is WT and the right bar is IAP-KO. The FITC-dextran test showed an age-dependent increase in gastrointestinal permeability, significantly influenced by IAP deficiency, as shown in FIG. 2C. Furthermore, expression levels of intestinal tight junction proteins were measured in ileal tissue of IAP-KO and WT animals. FIGS. 2D and 2E depict an association between age and loss of IAP with a significant reduction in expression levels of Occludin and ZO-1. Because gastrointestinal hyperpermeability can contribute to endotoxemia and local and systemic inflammation, proinflammatory cytokines and endotoxins were measured in ileal tissue and serum of IAP-KO and WT mice of different ages. As shown in FIGS. 2F-1I, inflammatory parameters and systemic endotoxin levels were significantly higher in older mice and in animals lacking IAP.

### Lack of IAP Associated with Aging-Related Liver Inflammation, Steatosis, and Increased Proinflammatory Characteristics of Portal Serum in Mice

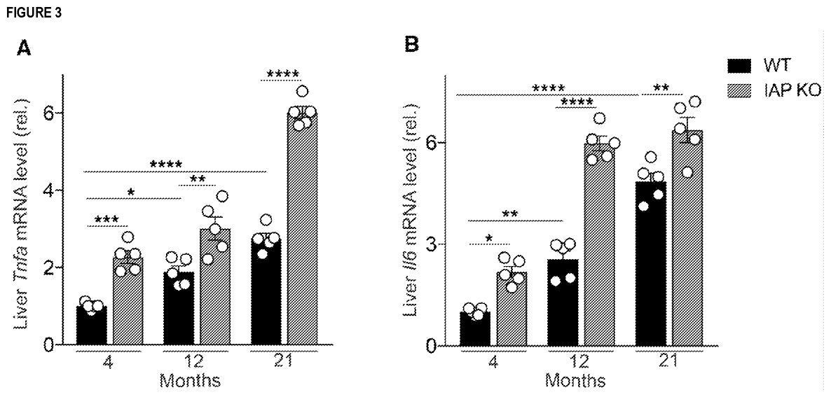

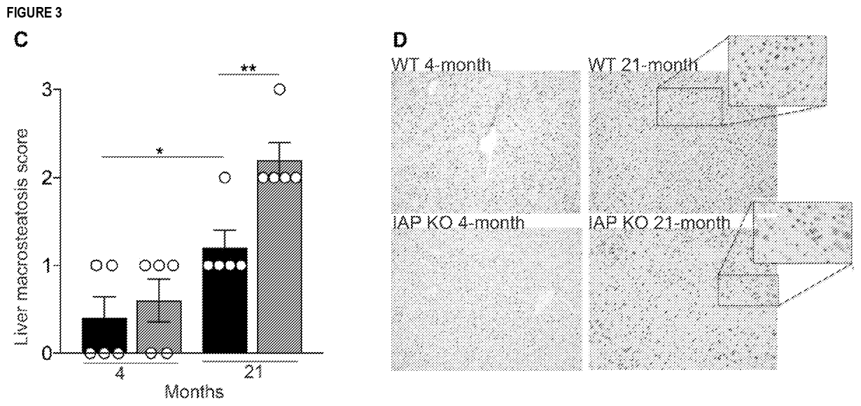

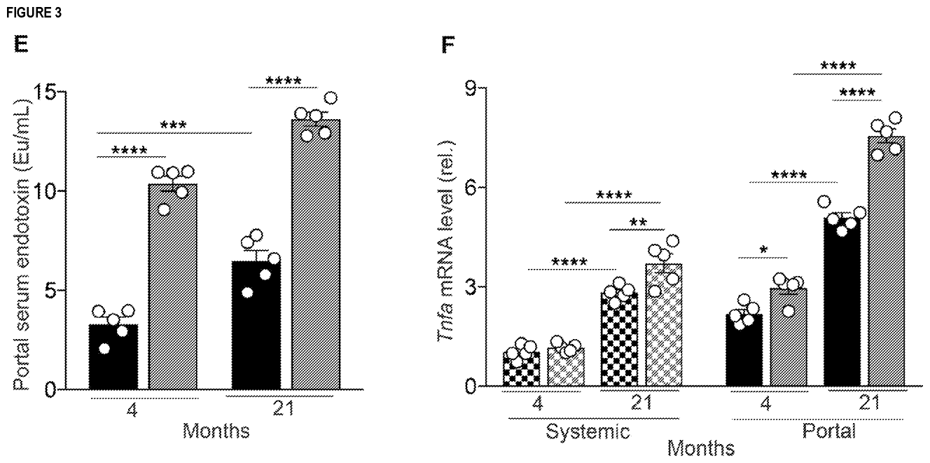

Next, it was evaluated whether there is an age-dependent increase in expression levels of the aging-associated, proinflammatory cytokines IL-6 and TNF in liver tissue. In each set of two histograms of FIGS. 3A-C, E-F, the left bar is WT and the right bar is IAP-KO. As FIGS. 3A and 3B show, a significant increase in expression levels of both cytokines IL-6 and TNF in liver tissue of WT mice was observed as a function of age. IAP-deficiency was associated with significantly increased expression levels of these proinflammatory cytokines IL-6 and TNF.

To examine age-related histologic changes and investigate the effects of IAP in such alterations, the liver, small intestine, and colon of young and old, WT, and IAP-KO mice were compared. For liver tissue, H&E and Oil Red O staining were performed. Within the liver, hepatocyte vacuolation, ballooning degeneration, inflammation, infiltration of predominantly lymphocytes, and scattered neutrophils were increased in old compared with the young WT mice. Furthermore, these changes in the liver were seen to a greater extent in aged IAP-KO mice. Marked microvesicular and macrovesicular steatosis were noted in both the 21-month-old WT and IAP-KO mice; however, more macrovesicular deposits were seen in the IAP-KO mice. Accordingly, as shown in FIG. 3C, the liver macrosteatosis score increased with age and was significantly higher in IAP-KO than in WT mice. The Oil Red O staining depicted in FIG. 3D illustrates the marked differences in neutral triglyceride and lipid deposits when young and old WT and IAP-KO mice were compared.

Because gastrointestinal-derived proinflammatory mediators contribute to liver inflammation, the impact of IAP on endotoxin dissemination was then investigated by measuring endotoxin levels in systemic and portal serum from young and old WT and IAP-KO mice by limulus amebocyte lysate (LAL) assay. FIG. 3E shows that the amount of LPS in portal and systemic serum increased significantly as a function of age but was greater than 1000 times higher in portal compared with systemic serum, regardless of age or genotype, consistent with its gastrointestinal source. The absence of IAP was associated with significantly more LPS in both portal and systemic blood (see FIG. 2I and FIG. 3E). To further determine the proinflammatory characteristics of portal and systemic serum from young and old IAP-KO and WT mice, the inflammatory response of target cells exposed to various sera were evaluated. Primary mouse BM-derived macrophages were incubated with portal and systemic serum from young and old WT and IAP-KO mice for 24 hours and Tnfa mRNA levels were then measured in the targeted cells. The results are shown in FIG. 3F, in which, upon incubation of target cells, it was found that both systemic and portal serum from old animals induced a significantly higher inflammatory response than serum derived from young animals. There was also a significant difference between the magnitude of Tnfa expression induced by portal compared with systemic serum. Finally, portal serum from IAP-KO mice resulted in a clearly more pronounced inflammatory response than serum from their WT counterparts.

### Example 2: IAP Supplementation Ameliorates Age-Induced Frailty and Age-Induced Physiological Alterations

The purpose of this experiment was to determine whether long-term IAP supplementation leads to reduced frailty and increased lifespan in mice, as well as having an effect on aging-induced gastrointestinal barrier dysfunction, endotoxemia, and chronic inflammation in mice.

### IAP Reduces Frailty and Extends Lifespan in Mice

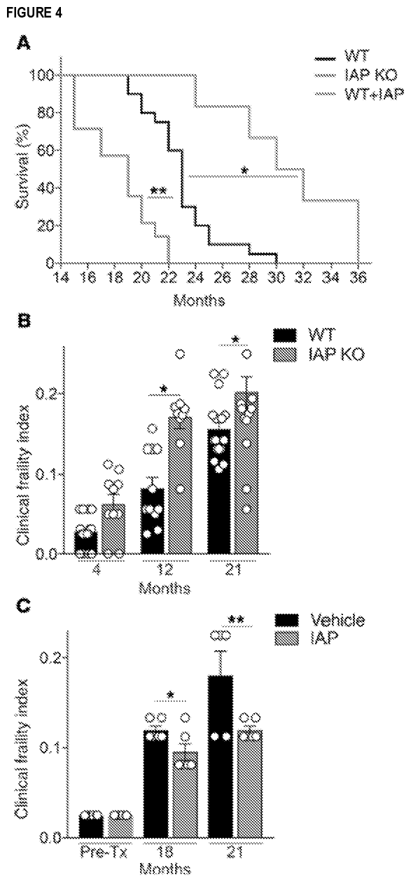

Given the impact of IAP deficiency on gastrointestinal barrier dysfunction, endotoxemia, and inflammation during aging, frailty and lifespan were also evaluated in IAP-KO mice, WT control mice (vehicle), and mice receiving IAP supplementation. IAP supplementation was started at the age of 10 months and led to significantly higher stool IAP activity levels. FIG. 4A-B shows that IAP deficiency was associated with a shorter lifespan (FIG. 4A) and more frailty (FIG. 4B), as compared to their WT littermates. At birth, IAP-deficient mice did not show any visible defects and were indistinguishable from their WT littermates. In addition, during the course of their lives, IAP-KO mice did not develop any significant gross abnormalities in appearance or fertility, including after breeding through multiple generations. As shown in FIG. 4B, no significant differences in frailty index existed among mice younger than 12 months. By contrast, when frailty was quantified in aged mice, significant differences were observed in older mice (>12 months), and IAP deficiency was associated with a significant difference in frailty, as depicted in FIG. 4B. There was also no difference in the frailty index of WT mice before starting IAP or vehicle supplementation, as shown in FIG. 4C. After 18 and 21 months, IAP supplementation led to marked differences in frailty; higher intestinal IAP activity levels were associated with lower frailty indices after 18 and 21 months of IAP compared with vehicle supplementation (FIG. 4C). Moreover, FIG. 4A depicts IAP-KO mice have a shortened lifespan and IAP supplementation in wild type mice led to a significantly extended lifespan compared with wild type mice receiving vehicle alone.

### IAP Ameliorates Aging-Induced Gastrointestinal Barrier Dysfunction, Endotoxemia, and Chronic Inflammation in Mice

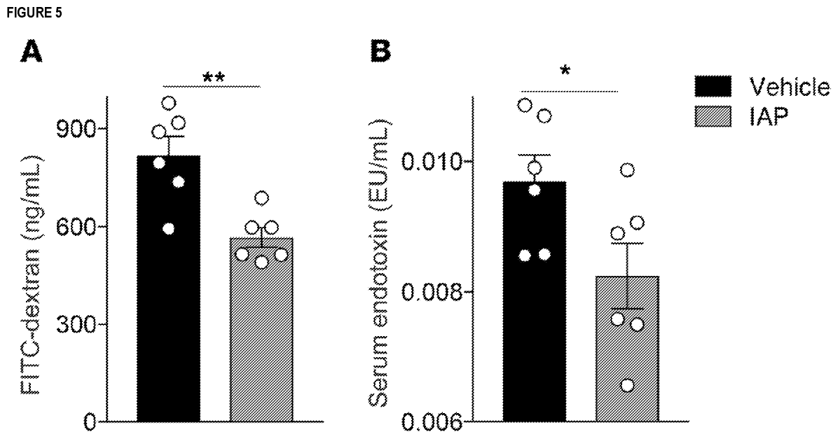

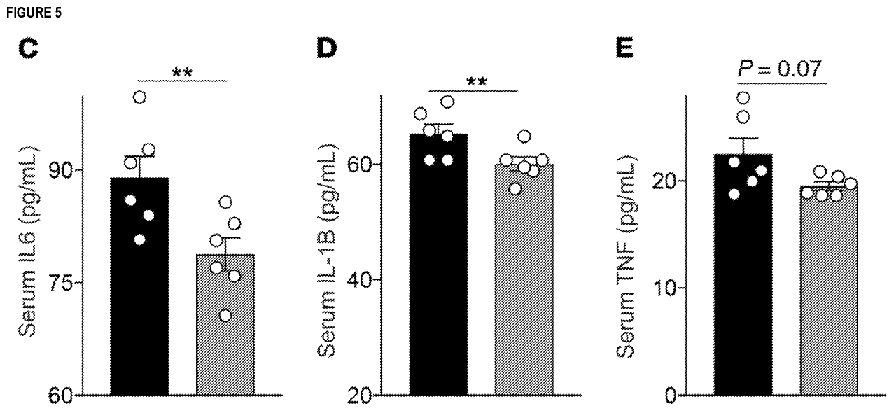

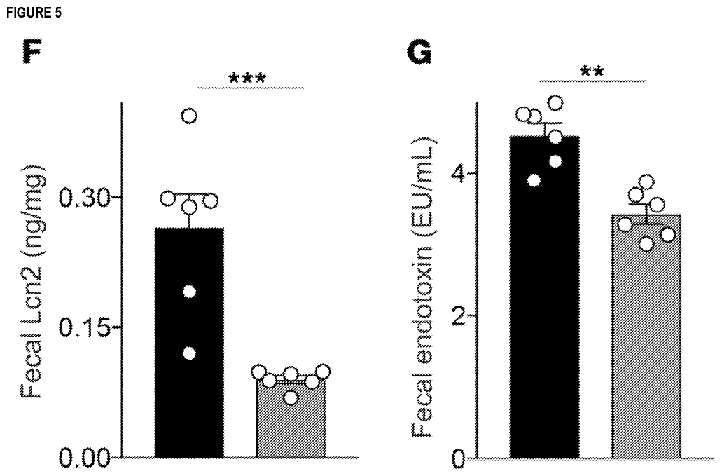

To further explore the effects of IAP supplementation on intestinal alterations in aging, gastrointestinal permeability was measured by a systemic serum FITC-dextran test in 21-month-old WT mice that had received IAP supplementation for 11 months. As depicted in FIG. 5A, gastrointestinal permeability was significantly reduced in mice supplemented with IAP, as compared with control animals. Next, blood serum endotoxin and cytokine levels in WT mice receiving IAP or vehicle were measured. Long-term IAP supplementation led to a significant reduction in endotoxin and proinflammatory cytokine levels compared with control mice, as shown in FIG. 5B-E. Furthermore, FIG. 5F shows that fecal Lipocalin 2 (Lcn2) levels, a sensitive marker for chronic (low-grade) intestinal inflammation, were measured in the stool of WT mice with or without IAP supplementation. Mice receiving IAP had significantly lower Lcn2, as well as fecal endotoxin levels, as depicted in FIG. 5F-G.

### Example 3: IAP Supplementation Improves Metabolic Profile and Aging-Related Gastrointestinal Microbiota Changes

The purpose of this experiment was to determine whether long-term IAP supplementation leads to an improved metabolic profile in wild-type (WT) mice, as well as whether IAP supplementation has an effect on aging-induced changes in gastrointestinal microbiota diversity.

### IAP Improves Metabolic Profile of WT Mice

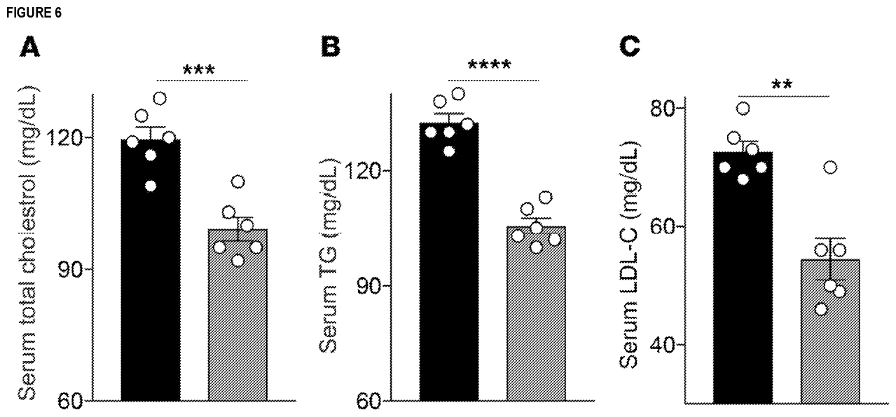

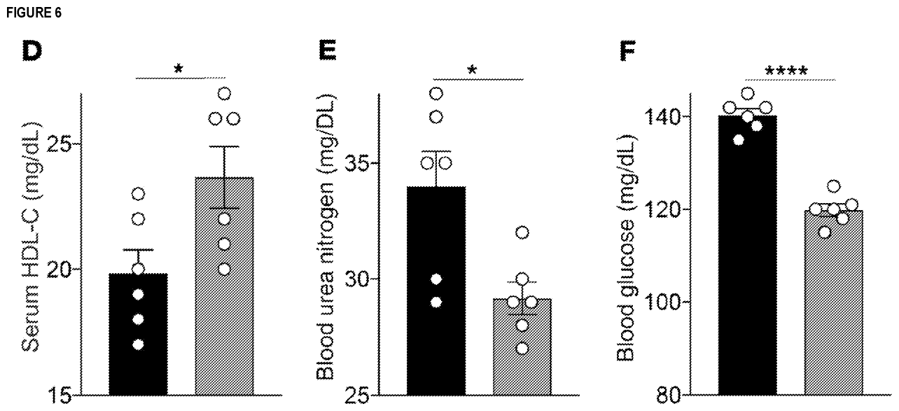

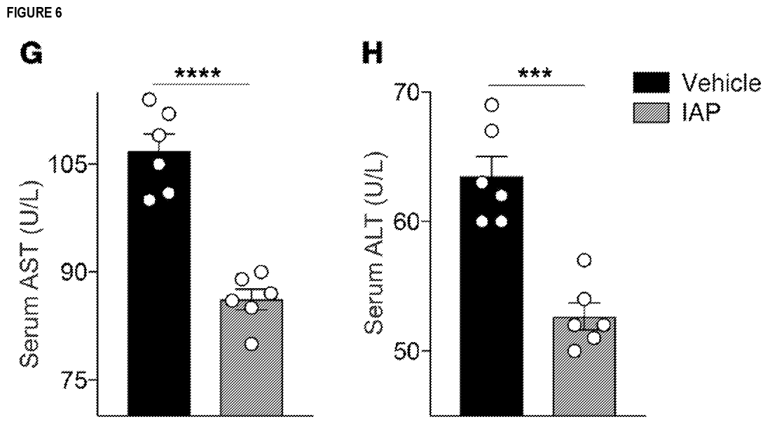

Given the IAP effects on gastrointestinal barrier function, endotoxemia, and systemic inflammation, next examined was the impact of IAP treatment on the metabolic profile in aging mice. FIG. 6A-F shows that long-term IAP treatment in mice receiving a standard chow diet was associated with a significantly improved lipid profile (FIG. 6A—D), as well as lower blood glucose and urea nitrogen levels, indicative of renal function (FIG. 6E-F). Furthermore, IAP treatment led to lower serum liver enzymes (FIG. 6G-H).

### IAP Inhibits Age-Related Compositional Changes of Gastrointestinal Microbiota

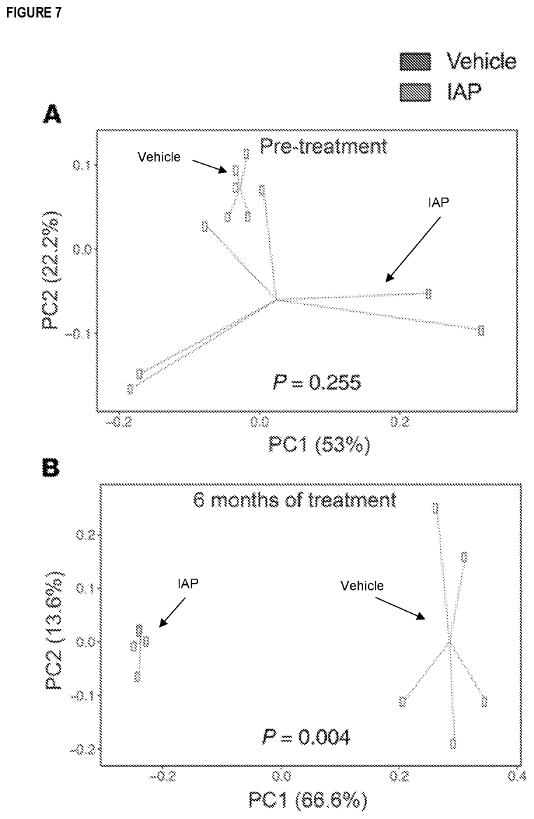

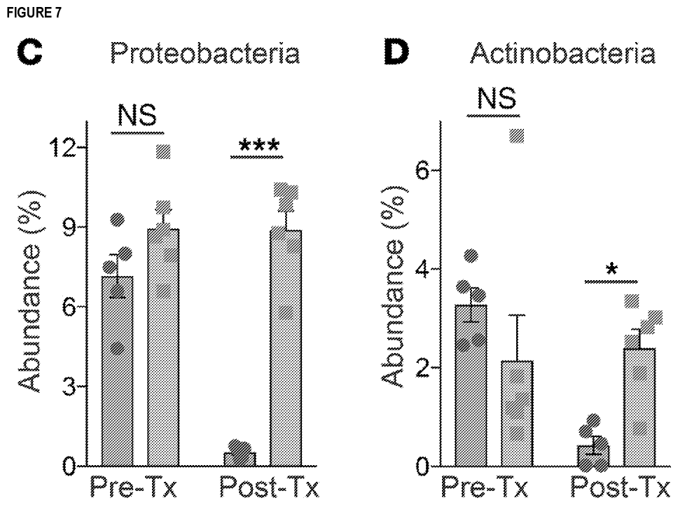

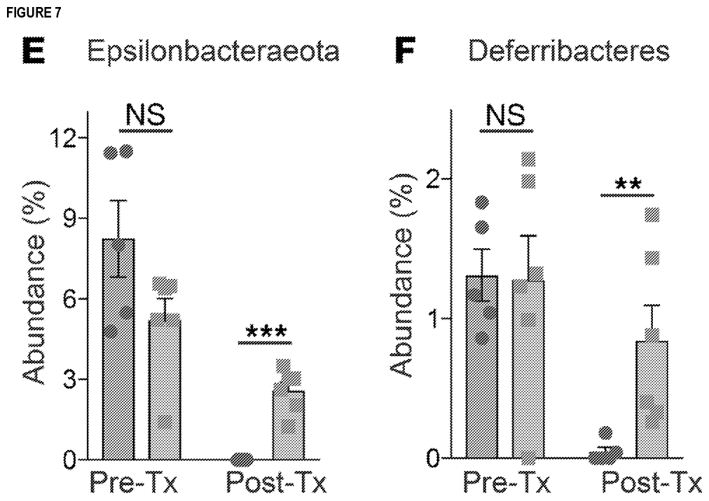

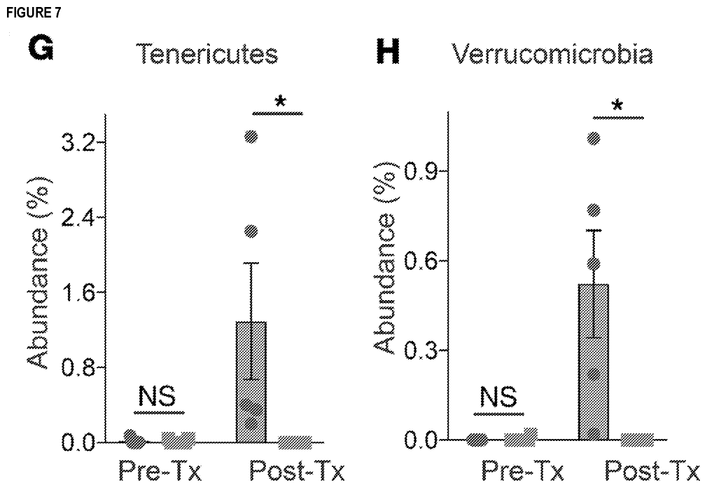

In order to determine the effect of supplemental oral IAP on age-associated changes in commensal bacterial populations, 16S rRNA sequencing and analysis of fecal samples were performed at various time points (before treatment, 1 month, and 6 months after treatment). As measured by observed operational taxonomic units (OTUs) and the Shannon diversity index, a diversity did not change over time for either vehicle-treated or IAP-treated mice. Then, Principal Component Analysis (PCA) on relative abundance of phyla at various time points and across treatment groups was performed. FIG. 7A-B shows that the PCA demonstrated that the first principal component, explaining 37.8% of the variation, separated the control group of vehicle-treated mice at the 6-month time point. The fecal samples from pretreatment, 1 month of treatment, and 6 months of IAP treatment mice all clustered together, indicating overall similarity in the phylum composition among these groups.

With regard to the relative abundance of specific phyla, it was observed that control mice demonstrated a clear change in microbiota phyla over the 6-month experiment. Specifically, a statistically significant decrease was observed in the abundances of Proteobacteria, Actinobacteria, Epsilonbacteraeota, and Deferribacteres as shown in FIG. 7C-F, while Tenericutes and Verrucomicrobia abundance was significantly increased, as depicted in FIG. 7G-H. No significant change was detected for the 2 most common phyla, Bacteroidetes and Firmicutes. In contrast, IAP-treated mice displayed minimal change in the relative abundance of phyla over time, with only a marginal increase in the abundance of Tenericutes at the 1-month time point, which reverted to pretreatment abundance after 6 months, as shown in FIG. 7G. When comparing the relative abundance of phyla between IAP- and control-treated mice after 6 months, the abundance of the 2 most common phyla were similar; however, vehicle-treated mice had significantly less Proteobacteria, Actinobacteria, Epsilonbactareota, and Deferribacteres and significantly more Verrucomicrobia. Taken together, the data demonstrate that long-term IAP treatment appears to inhibit the age-associated change in fecal microbial composition over time.

### Equivalents

While the disclosure has been described in connection with specific embodiments thereof, it will be understood that it is capable of further modifications and this disclosure is intended to cover any variations, uses, or adaptations of the disclosure following, in general, the principles of the disclosure and including such departures from the present disclosure as come within known or customary practice within the art to which the disclosure pertains and as may be applied to the essential features set forth herein and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### Incorporation by Reference

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### Other Embodiments

A number of embodiments of the disclosure have been described. Nevertheless, it will be understood that various modifications may be made without departing from the spirit and scope of the disclosure. Accordingly, other embodiments are within the scope of the following claims.

What is claimed is:

1. A method of treating an age-related physiological alteration of, or related to, intestinal homeostasis in a subject in need thereof, the method comprising administering an alkaline phosphatase (AP)-based agent to the subject.

2. The method of claim 1, wherein the age-related physiological alteration of, or related to, intestinal homeostasis is selected from one or more of increased gastrointestinal permeability, increased gastrointestinal-derived systemic inflammation, increased chronic inflammation, increased gastrointestinal barrier dysfunction, dysbiosis, endotoxemia, and increased levels of proinflammatory cytokines or chemokines.

3. The method of claim 1 or claim 2, wherein the age-related physiological alteration of, or related to, intestinal homeostasis is measured by a decrease in ZO-1 protein, ZO-2 protein, occludin, or tight junction proteins, or is measured by an increase in HMGB1 (High Mobility Group Box 1).

4. The method of claim 2, wherein the proinflammatory cytokine is selected from one or more of Interleukin 6 (IL-6), Tumor Necrosis Factor-alpha (TNF-α), Interleukin 1 (IL-1), Interleukin 8 (IL-8), and Interleukin 18 (IL-18).

5. The method of claim 2, wherein the proinflammatory chemokine is selected from one or more of C-Reactive Protein (CRP) and Macrophage-Derived Chemokine 2 (MDC-2).

6. The method of any one of the preceding claims, wherein the age-related physiological alteration of, or related to, intestinal homeostasis is associated with frailty and/or a decreased lifespan.

7. The method of any one of the preceding claims, wherein the age-related physiological alteration of, or related to, intestinal homeostasis is associated with an age-related disorder and/or the subject is afflicted with said age-related disease or disorder.

8. The method of claim 7, wherein the age-related disease or disorder is selected from kidney failure, liver inflammation, steatosis, hepatic steatosis, non-alcoholic fatty liver disease (NAFLD), non-alcoholic steatohepatitis (NASH), type 2 diabetes, hepatocellular carcinoma, atherosclerotic cardiovascular disease (ASCVD), cachexia, metabolic syndrome, osteoarthritis, inflammatory bowel disease (IBD), and Alzheimer's disease.

9. A method of treating age-related frailty in a subject in need thereof, the method comprising administering an AP-based agent to the subject.

10. The method of claim 9, wherein frailty comprises an accumulation of deficiencies in major physiological functions, reduction of regeneration capabilities, impaired wound healing, and/or increased risk of age-related diseases or disorders.

11. The method of claim 9 or claim 10, further comprising measuring the age-related frailty using a Physiological Frailty Index.”

12. The method of claim 11, wherein the Physiological Frailty Index comprises assessment of one or more parameters selected from grip strength, systolic blood pressure, diastolic blood pressure, blood flow volume, number of blood neutrophils, percentage of blood neutrophils, number of blood monocytes, percentage of blood monocytes, number of lymphocytes, number of red blood cells, hemoglobin levels, hematocrit levels, mean corpuscular volume, mean corpuscular hemoglobin levels, mean corpuscular hemoglobin concentration and keratinocyte-derived cytokine levels.

13. The method of claim 11 or claim 12, wherein the Physiological Frailty Index score of a subject receiving the AP-based agent is improved or is lower than the Physiological Frailty Index score of a subject not receiving the AP-based agent.

14. The method of claim 13, wherein the subject's Physiological Frailty Index score is reduced by about 25% to about 75%.

15. The method of claim 13, wherein the subject's Physiological Frailty Index is reduced by at least about 75%, or about 50%, or about 35%, or about 25%.

16. A method of inhibiting or reversing an age-related change of gastrointestinal microbiota phylum diversity in a subject in need thereof, the method comprising administering an AP-based agent to the subject.

17. The method of claim 16, wherein the gastrointestinal microbiota phylum is selected from one or more of Proteobacteria, Actinobacteria, Epsilonbactareota, Deferribacteres, Tenericutes, and Verrucomicrobia.

18. The method of claim 16 or claim 17, wherein the age-related change of gastrointestinal microbiota phylum diversity is a decrease in the abundance of the microbiota phylum selected from one or more of Proteobacteria, Actinobacteria, Epsilonbactareota, and Deferribacteres.

19. The method of claim 16 or claim 17, wherein the age-related change of gastrointestinal microbiota phylum diversity is an increase in the abundance of the microbiota phylum selected from Tenericutes and Verrucomicrobia.

20. The method of any one of claims 16-19, wherein administering the AP-based agent to the subject in need thereof results in gastrointestinal microbiota phylum diversity similar to the phylum diversity exhibited by a subject not having an age-related change.

21. The method of any one of claims 16-20, wherein the gastrointestinal microbiota phylum diversity is measured by performing 16s rRNA sequencing of a sample from the subject.

22. The method of claim 21, wherein the sample is a fecal sample.

23. The method of claim 21 or claim 22, wherein the measurement of the subject's gastrointestinal microbiota phylum diversity is an operational taxonomic unit (OTU) or a value of the Shannon diversity index.

24. The method of any one of the preceding claims, wherein the subject is a human patient.

25. The method of claim 24, wherein the human patient is elderly.

26. The method of claim 25, wherein the human patient is between about 56 and about 85 years old.

27. The method of claim 25, wherein the human patient is equal to or older than about 65 years old.

28. The method of claim 24, wherein the human patient is middle-aged.

29. The method of claim 28, wherein the human patient is between about 36 and about 55 years old.

30. A method of treating an age-related physiological alteration of, or related to, intestinal homeostasis in a non-elderly subject in need thereof, the method comprising
screening the non-elderly subject for one or more age-related physiological alterations of, or related to, intestinal homeostasis selected from one or more of increased gastrointestinal permeability, increased gastrointestinal-derived systemic inflammation, increased chronic inflammation, increased gastrointestinal barrier dysfunction, dysbiosis, endotoxemia, and increased proinflammatory cytokines or chemokines, and
administering to the subject an AP-based agent when the screen indicates the one or more physiological alterations are associated with aging.

31. The method of claim 30, wherein the screen for the one or more age-related physiological alterations is selected from a decrease in ZO-1 protein, a decrease in ZO-2 protein, a decrease in occludin, a decrease in tight junction proteins, and an increase in HMGB1 (High Mobility Group Box 1).

32. A method of treating and/or delaying the onset of accelerated aging in a subject in need thereof, the method comprising administering an alkaline phosphatase (AP)-based agent to the subject.

33. The method of claim 32, wherein the accelerated aging is a progeroid syndrome, or symptom thereof.

34. The method of claim 33, wherein the progeroid syndrome is selected from Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), and restrictive dermopathy (RD).

35. The method of any one of claims 30-34, wherein the subject is middle-aged.

36. The method of claim 35, wherein the subject is between about 36 and about 55 years old.

37. The method of any one of the preceding claims, wherein the AP-based agent is administered enterally or parenterally.

38. The method of claim 37, wherein the enteral administration is oral administration.

39. The method of any one of the preceding claims, wherein the AP-based agent comprises intestinal alkaline phosphatase (IAP).

40. The method of claim 39, wherein the IAP comprises bovine IAP (bIAP).

41. The method of claim 40, wherein the bIAP comprises an amino sequence having at least about 90%, or about 95%, or about 97%, or about 98%, or about 99% sequence identity to one of SEQ ID NO: 1 to SEQ ID NO: 11.

42. The method of claim 41, wherein the bIAP comprises an amino sequence having at least about 97% sequence identity to one of SEQ ID NO: 1 to SEQ ID NO: 11.

43. A composition comprising an alkaline phosphatase (AP)-based agent for use in a method of any one of claims 1 to 42.

44. The composition of claim 43, wherein the AP-based agent comprises intestinal alkaline phosphatase (IAP).

45. The composition of claim 44, wherein the IAP comprises bovine IAP (bIAP).

46. The composition of claim 45, wherein the bIAP comprises an amino sequence having at least about 90%, or about 95%, or about 97%, or about 98%, or about 99% sequence identity to one of SEQ ID NO: 1 to SEQ ID NO: 11.

47. The composition of claim 46, wherein the bIAP comprises an amino sequence having at least about 97% sequence identity to one of SEQ ID NO: 1 to SEQ ID NO: 11.


SEQUENCE LISTING (12 sequences)

SEQ ID NO: 1 - protein, 528 aa, Artificial Sequence
  Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 2 - protein, 533 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDAAHLAASPPPLALLAGAMLLLLAPTLY

SEQ ID NO: 3 - protein, 503 aa, Artificial Sequence
  Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPAGTTD

SEQ ID NO: 4 - protein, 506 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 5 - protein, 754 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 534 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLAPPAGTTDAAHPIEGRSVVPALLPLRAGTLLLLETATAP

SEQ ID NO: 7 - DNA, 1669 bp, Artificial Sequence
  Synthetic polymer.
atgcaggggccctgggtgctgctgctgctgggcctgaggctacagctctccctgggcgtcatcccaggtaatgaggctccccaagctgttccacacacagggcaccccctcagccaggctgacctgatctctactctccccctggccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 8 - DNA, 1671 bp, Artificial Sequence
  Synthetic polymer.
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctgatttactaaaaccttgaaataaaattgtaaaacatcagtttgaaggcctgactctcagggtagttcttttttaattctgggtttt

SEQ ID NO: 9 - DNA, 1259 bp, Artificial Sequence
  Synthetic polymer.
aaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgaacaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcaggaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaa

SEQ ID NO: 10 - protein, 420 aa, Artificial Sequence
  Synthetic polymer.
KKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 11 - protein, 487 aa, Artificial Sequence
  Synthetic polymer.
LIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 12 - DNA, 13 bp, Artificial Sequence
  Synthetic polymer
gccgccaccatgg